SEQUENCE LISTING (19 sequences)

SEQ ID NO: 1 - DNA, 20 bp, Homo sapiens
  source 1..20: mol_type = other DNA
gtctgagcttccagatgaga

SEQ ID NO: 2 - DNA, 20 bp, Homo sapiens
  source 1..20: mol_type = other DNA
aggagcatcttgaggatgga

SEQ ID NO: 3 - DNA, 20 bp, Homo sapiens
  source 1..20: mol_type = other DNA
catgagtgtgactgctggtt

SEQ ID NO: 4 - DNA, 20 bp, Homo sapiens
  source 1..20: mol_type = other DNA
tgttgatggtcatgtcatgc

SEQ ID NO: 5 - DNA, 20 bp, Homo sapiens
  source 1..20: mol_type = other DNA
tggatccagacaactgttca

SEQ ID NO: 6 - DNA, 20 bp, Homo sapiens
  source 1..20: mol_type = other DNA
gagtccgggcaggtctactt

SEQ ID NO: 7 - DNA, 20 bp, Homo sapiens
  source 1..20: mol_type = other DNA
cagcctgagttgttgatgat

SEQ ID NO: 8 - DNA, 20 bp, Homo sapiens
  source 1..20: mol_type = other DNA
atgtccgtcatagtggtgga

SEQ ID NO: 9 - DNA, 20 bp, Homo sapiens
  source 1..20: mol_type = other DNA
ggagctttggctgtgtacta

SEQ ID NO: 10 - DNA, 20 bp, Homo sapiens
  source 1..20: mol_type = other DNA
gctgaaggaccttgttgttt

SEQ ID NO: 11 - DNA, 18 bp, Mus musculus
  source 1..18: mol_type = other DNA
agtcatcaccatgtttct

SEQ ID NO: 12 - DNA, 18 bp, synthetic construct
  source 1..18: mol_type = other DNA
  modified_base order(1,2,3,16,17,18): mod_base = OTHER; note = 2' O-methyl RNA
  modified_base order(1,2): mod_base = OTHER; note = phosphorothioate linkage
  modified_base order(17,18): mod_base = OTHER; note = phosphorothioate linkage
agtcatcaccatgtttct

SEQ ID NO: 13 - DNA, 18 bp, synthetic construct
  source 1..18: mol_type = other DNA
  modified_base order(1,2,3,16,17,18): mod_base = OTHER; note = 2' O-methly RNA
  modified_base order(1,2): mod_base = OTHER; note = phosphorothioate linkage
  modified_base order(17,18): mod_base = OTHER; note = phosphorothioate linkage
agtcatcaccatgtttct

SEQ ID NO: 14 - DNA, 18 bp, synthetic construct
  source 1..18: mol_type = other DNA
  modified_base order(1,2,3,16,17,18): mod_base = OTHER; note = 2' O-methyl RNA
  modified_base order(1,2): mod_base = OTHER; note = phosphorothioate linkage
  modified_base order(17,18): mod_base = OTHER; note = phosphorothioate linkage
agtcatcaccatgtttct

SEQ ID NO: 15 - DNA, 18 bp, synthetic construct
  source 1..18: mol_type = other DNA
  modified_base order(1,2,3,16,17,18): mod_base = OTHER; note = 2' O-methyl RNA
  modified_base order(1,2): mod_base = OTHER; note = phosphorothioate linkage
  modified_base order(17,18): mod_base = OTHER; note = phosphorothioate linkage
gtttcaccacccaattcc

SEQ ID NO: 16 - DNA, 18 bp, synthetic construct
  source 1..18: mol_type = other DNA
  modified_base 1..18: mod_base = OTHER; note = locked nucleic acid
gctattaccttaacccag

SEQ ID NO: 17 - protein, 15 aa, synthetic construct
  MOD_RES 2: note = 6-aminohexanoic acid
  MOD_RES 8: note = 6-aminohexanoic acid
  MOD_RES 13: note = 6-aminohexanoic acid
  MOD_RES 15: note = 6-aminohexanoic acid
  MOD_RES 5: note = bAla
  MOD_RES 11: note = bAla
  MOD_RES 14: note = bAla
RXRRBRRXRRBRXBK

SEQ ID NO: 18 - DNA, 23 bp, synthetic construct
  source 1..23: mol_type = other DNA
cgtaaacggccacaagttcagcg

SEQ ID NO: 19 - DNA, 24 bp, synthetic construct
  source 1..24: mol_type = other DNA
gtggtgcagatgaacttcagggtc

(19) United States
(12) Patent Application Publication
(10) Pub. No.: US 2025/0332270 A1
(43) Pub. Date: Oct. 30, 2025

(54) Targeting Skin-Related Conditions Using Bottlebrush Polymer-Conjugated Oligonucleotides

(71) Applicant: Northeastern University, Boston, MA (US)

(72) Inventors: Yang Fang, Boston, MA (US); Ke Zhang, Natick, MA (US)

(21) Appl. No.: 19/195,709

(22) Filed: Apr. 30, 2025

Related U.S. Application Data
(60) Provisional application No. 63/640,851, filed on Apr. 30, 2024.

(51) Int. Cl. A61K 47/60; A61K 9/00; A61P 17/06; C12N 15/113

(52) U.S. Cl. CPC A61K 47/60; A61K 9/0019; A61P 17/06; C12N 15/1136; C12N 2310/11; C12N 2310/315; C12N 2310/321; C12N 2310/322; C12N 2310/3231; C12N 2310/351; C12N 2320/32; C12N 2320/33

(57) ABSTRACT

Provided herein are, in various embodiments, methods and compositions for treating a skin-related condition in a subject in need thereof, comprising administering to the subject a therapeutically effective amount of a composition comprising a bottlebrush polymer-oligonucleotide conjugate. Also provided herein are methods of making bottlebrush polymer-oligonucleotide conjugates.

### RELATED APPLICATIONS

This application claims the benefit of U.S. Provisional Application No. 63/640,851, filed Apr. 30, 2024. The entire teachings of the above application are incorporated herein by reference.

### GOVERNMENT SUPPORT

This invention was made with government support under Grant Nos. 1R01CA251730, 1R01GM121612-01, and 42CA275425 awarded by the National Institutes of Health, and under Grant No. 2004947 awarded by the National Science Foundation. The government has certain rights in the invention.

### INCORPORATION BY REFERENCE OF MATERIAL IN XML

This application incorporates by reference the Sequence Listing contained in the following extensible Markup Language (XML) file being submitted concurrently herewith:
a) File name: 5200.2400-001.xml; created Apr. 30, 2025, 27,000 Bytes in size.

### BACKGROUND

To date, the development of oligonucleotides therapeutics has been concentrated on a limited set of organs and tissues including the liver, skeletal muscle, and the central nervous system. While the skin is the largest vascularized organ of the body, the distribution and retention of intravenously (i.v.) injected oligonucleotide formulations in the skin is not as well studied. The lack of interest in the skin may be in part due to the much lower content of blood circulating oligonucleotides absorbed in the skin compared to the kidney, liver, and spleen, as suggested by current studies. Accordingly, effective agents are needed to realize skin delivery of oligonucleotide therapeutics.

### SUMMARY

In one aspect, the disclosure provides a method for treating or preventing a skin-related condition in a subject in need thereof. The method comprises administering to the subject a therapeutically effective amount of a composition comprising a bottlebrush polymer-oligonucleotide conjugate. In some embodiments, the conjugate comprises a polymer backbone, polyethylene glycol (PEG) polymer arms covalently linked to the polymer backbone, and an oligonucleotide covalently linked to the backbone. The oligonucleotide modulates expression of one or more transcripts associated with the skin-related condition.

In some embodiments, the skin-related condition is selected from autoimmune skin diseases, skin cancers or precancers, pigmentary disorders, genetic skin disorders, inflammatory skin disorders, infectious skin diseases, wound healing disorders, and scarring disorders.

In some embodiments, the oligonucleotide modulates gene expression by mRNA degradation, translation inhibition, splice modulation, RNA editing, gene activation via RNA activation (RNAa), or any combination thereof.

In some embodiments, the oligonucleotide comprises a sequence complementary to a region of the one or more transcripts associated with the skin-related condition. In some embodiments, the one or more transcripts comprise a human transcript selected from: interleukin-17A (IL-17A); interleukin-17A receptor (IL-17RA); interleukin-23 (IL-23); interleukin-17F (IL-17F); interleukin-17C receptor (IL-17RC); tumor necrosis factor-alpha (TNF-α); interferon-gamma (IFN-γ); Janus kinase 1 (JAKI); Janus kinase 3 (JAK3); signal transducer and activator of transcription 3 (STAT3); tyrosinase (TYR); microphthalmia-associated transcription factor (MITF); collagen type I alpha 1 (COL1A1); matrix metalloproteinase 9 (MMP9); cyclin dependent kinase inhibitor 2A (CDKN2A); B-Raf proto-oncogene, serine/threonine kinase (BRAF); or any combination thereof. In some embodiments, the sequence complementary to the region of the disease-associated transcript comprises any one of SEQ ID NOs: 1-10.

In some embodiments, the oligonucleotide comprises a chemically modified nucleic acid. In some embodiments, the chemically modified nucleic acid comprises one or more locked nucleic acid (LNA) modified bases, one or more phosphorothioate internucleotide linkages, one or more RNA bases with 2′ modifications, or any combination thereof.

In some embodiments, the polymer backbone comprises two or more monomers selected from: a synthetic monomer selected from a serinol, a norbornene, an acrylate, and an acrylamide; a natural monomer selected from an amino acid and a sugar; a modified form of a natural molecule selected from a morpholino phosphorodiamidate, a modified amino acid, a modified spermine, a modified lipid, and a modified cholesterol; or any combination thereof.

In some embodiments, the bottlebrush polymer-oligonucleotide conjugate comprises about 30 PEG polymer arms; each of the PEG polymer arms is about 10 kDa; or both of the foregoing.

In some embodiments, the composition is administered to the subject as a systemic injection. In some embodiments, the composition is administered to the subject as a dose of about 0.5 mg oligonucleotide per kg bodyweight (0.5 mg/kg) to about 5 mg oligonucleotide per kg bodyweight (5 mg/kg). In some embodiments, the composition is administered to the subject via the subject's skin. In some embodiments, the composition is administered to the subject topically, intradermally, or transdermally.

In some embodiments, the bottlebrush polymer-oligonucleotide conjugate further comprises a targeting ligand.

In another aspect, the disclosure provides a bottlebrush polymer-oligonucleotide conjugate, comprising: a polymer backbone; polyethylene glycol (PEG) polymer arms covalently linked to the polymer backbone; and an oligonucleotide covalently linked to the backbone, wherein the oligonucleotide modulates expression of one or more transcripts associated with the skin-related condition. In yet another aspect, the disclosure provides a composition comprising the bottlebrush polymer-oligonucleotide conjugate and one or more pharmaceutically acceptable excipients, diluents, or carriers suitable for topical, transdermal, intradermal, or systemic administration.

In yet another aspect, the disclosure provides a method for diagnosing a skin-related condition in a subject in need thereof, comprising: (1) scoring or measuring a parameter related to the skin-related condition in the subject, thereby obtaining a pre-treatment level of the parameter; (2) administering to the subject a therapeutically effective amount of a composition comprising a bottlebrush polymer-oligonucleotide conjugate, wherein the conjugate comprises: a polymer backbone, polyethylene glycol (PEG) polymer arms covalently linked to the polymer backbone, and an oligonucleotide covalently linked to the backbone, wherein the oligonucleotide modulates expression of one or more transcripts associated with the skin-related condition; (3) scoring or measuring the parameter related to the skin-related condition in the subject, thereby obtaining a post-treatment level of the parameter; and (4) comparing the post-treatment level to the pre-treatment level, wherein a difference between the post-treatment level and the pre-treatment level indicates presence of the skin-related condition in the subject.

In yet another aspect, the disclosure provides a method for improving an aspect of skin of a subject, comprising administering to the subject a composition comprising a bottlebrush polymer-oligonucleotide conjugate, wherein the conjugate comprises: a polymer backbone; polyethylene glycol (PEG) polymer arms covalently linked to the polymer backbone; and an oligonucleotide covalently linked to the backbone, wherein the oligonucleotide modulates expression of one or more transcripts associated with the skin-related condition. In some embodiments, the aspect comprises improving the appearance of skin in a subject. In some embodiments, the aspect comprises one or more of pigmentation irregularities, uneven skin tone, rough or dry skin texture, fine lines, wrinkles, and scars.

### BRIEF DESCRIPTION OF THE DRAWINGS

The patent or application file contains at least one drawing executed in color. Copies of this patent or patent application publication with color drawings will be provided by the Office upon request and payment of the necessary fec.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1B. Scheme illustrating example synthesis of polymer-assisted compaction of DNA (pacDNA)-1, pacDNA-2, Cy5-labeled pacDNA-1, and pacDNA-654. FIG. 1A) To synthesize the diblock bottlebrush polymer, a sequential ring-opening metathesis polymerization (ROMP) reaction was employed using two monomers: 7-oxanorbornenyl bromide (N-Br; compound 1) and norbornenyl-modified polyethylene glycol (PEG) (N-PEG; compound 2). The reaction yielded a diblock architecture (pNBr₅-bpNPEG₂₉) (compound 3). FIG. 1B) Next, azide substitution of bromides (yielding compound 4) and subsequent coupling with dibenzocyclooctyne (DBCO)-modified antisense oligonucleotide (ASO) strands (yielding compound 5) was carried out.

FIG. 2. N,N-dimethylformamide gel permeation chromatography (DMF-GPC) chromatogram of the bottlebrush polymer.

FIGS. 3A-3D. Physiochemical characterization of pacDNA. FIG. 3A) Schematic of chemical structure of pacDNA. FIG. 3B) Aqueous gel permeation chromatogram of pacDNA-1.

FIG. 3C) Agarose gel electrophoresis (2%) free antisense oligonucleotide (ASO) and pacDNA counterparts. FIG. 3D) Zeta potential measurements of gapmer-1 and pacDNA-1 in NANOPURE® bacterial- and particle-free ultrapure water.

FIGS. 4A-4D. Biodistribution and target engagement in mice. FIG. 4A) Ex vivo image of mouse dorsal skin and other major organs 24 hours post tail vein injection of cyanine 5 (Cy5)-bottlebrush polymer. FIG. 4B) Graph of quantitative biodistribution determined using tissue homogenate. FIG. 4C) Representative fluorescence micrograph of mouse dorsal skin cryosection (embedded in optimal cutting temperature compound (O.C.T.)) 24 hours after injection (Cy5 label resides on ASO component). Cell nuclei were stained with Hoechst 33342 DNA-specific fluorescent stain. FIG. 4D) Image of electrophoresis gel showing modified enhanced green fluorescence protein (EGFP) transcript isolated from skin tissue of EGFP-654 mice after treatment as determined by RT-PCR. Bands shown as “Ab” (160 bp) and “Co” (87 bp) represent aberrantly spliced and correctly spliced EGFP mRNA, respectively.

FIG. 5. Graph of quantification of the fluorescence signals of Cy5-labeled brush polymer in ex vivo skin and major organs to determine biodistribution.

FIG. 6. Fluorescence imaging of various tissue homogenates to explore biodistribution of Cy5-labeled brush polymer in mice.

FIG. 7. Ex vivo imaging of mouse dorsal skin 24 hours post tail vein injection of Cy5-labeled free gapmer-1 and pacDNA-1.

FIG. 8. Fluorescence micrographs of mouse dorsal skin frozen section (OCT-embedded) 24 hours after intravenous (i.v.) injection of Cy5-labeled brush polymer (red). Cell nuclei are stained with Hoechst 33342 (blue). Bright field (right micrograph).

FIGS. 9A-9D. Fluorescence micrographs of immunofluorescence-stained normal mouse skin tissue sections 24 hours after i.v. injection of Cy5-labeled brush polymer (red). Alexa Fluor 555-conjugated anti-rabbit IgG antibody served as secondary antibody (yellow). Cell nuclei were stained with Hoechst 33342 (blue). FIG. 9A) Dermal macrophages stained with F4/80 primary antibody. FIG. 9B) Dermal dendritic cells stained with CD11c primary antibody.

FIG. 9C) Fibroblasts stained with vimentin primary antibody. FIG. 9D) Adipose cells stained with perilipin-1 primary antibody or bright field only. Representative con-localization of stained cells and nanoparticles is indicated with yellow arrows.

FIG. 10. Fluorescence micrograph of mouse dorsal skin frozen section (OCT-embedded) 24 hours after i.v. injection of Cy5-labeled pacDNA (red). Cell nuclei were stained with Hoechst 33342 (blue).

FIG. 11. Schematic showing the modified EGFP gene in EGFP-654 transgenic mice that were treated with pacDNA-654, B pep-654, or vehicle. EGFP: green. Human β-globin IVS2-654 intron: yellow. ASO: blue.

FIG. 12. Schedule of EGFP-654 mice treatment with pacDNA-654 at dosage of 1.0 μmol/kg, B pep-654 at dosage of peptide 1.0 μmol/kg, or vehicle only. Mice were sacrificed at one week post last injection.

FIGS. 13A-13D. In vitro uptake and interleukin-17A receptor (IL-17RA) silencing. FIG. 13A) A graph quantifying cellular uptake of Cy5-labeled pacDNA, unmodified DNA ASO, and gapmer-1 by NIH/3T3 mouse embryonic fibroblast cells (0.1 to 2 μm ASO, 4 hours) as determined by flow cytometry. FIG. 13B) Graph quantifying dose-dependent reduction of IL-17RA in RAW 264.7 macrophage-like mouse cells by pacDNA-1 (72 hour treatment). FIGS. 13C, 13D) Western blot analysis of IL-17RA protein levels in RAW 264.7 and NIH/3T3 cells after treatment with pacDNA-1, pacDNA-2, or bottlebrush polymer (72 hours). *p<0.05, **p<0.01, ***p<0.001, ****p<0.0001 (two-tailed test).

FIG. 14. Flow cytometry measurement of NIH/3T3 cells treated with Cy5-labeled pacDNA-1, free gapmer-1 ASO, or free unmodified ASO (0.1 to 2 μM, ASO basis, 4 hours).

FIG. 15. Schedule of treatment for imiquimod (IMQ) topical application and pacDNA or control (phosphate-buffered saline (PBS)) injection in psoriasis mouse model.

FIGS. 16A-16E. In vivo phenotypic response. FIG. 16A) Representative images of mouse dorsal skin of each treatment group. FIG. 16B) Disease progression as indicated by Psoriasis Area and Severity Index (PASI) score (cumulative score of thickness plus erythema plus scaling, score: 0-12) as assessed by six blinded reviewers. FIGS. 16C-16E) Individual scoring of redness (FIG. 16C), thickness (FIG. 16D), and scaling (FIG. 16E) on a scale from 0-4.

FIG. 17. Redness levels (a* value) of mouse dorsal skin were recorded by tristimulus colorimeter daily.

FIGS. 18A-18E. Biochemical and histological analysis. FIG. 18A) Representative hematoxylin and eosin (H&E) and FIG. 18B) Ki67 proliferation marker immunohistochemical staining of mouse dorsal skin collected on day 7. Blank, no IMQ induction. FIG. 18C) Average epidermal thickness measured from multiple H&E-stained images. FIG. 18D) The number of epidermal Ki67-positive cells of each group counted in five areas. ****p<0.0001 (two-tailed test). FIG. 18E) Expression levels of IL-17RA in the dorsal skin of IMQ-induced mouse with injection of pacDNA-1, pacDNA-2, or vehicle only (control IMQ) as determined by western blot.

FIG. 19. Representative immunohistochemical staining for IL-17RA in day 7-harvest mouse dorsal skin from different treatment groups. Scale bar: 100 μm.

FIGS. 20A-20C. The mRNA expression of several psoriasis markers including FIG. 20A) (Serpina1c (Pi3), FIG. 20B) tumor necrosis factor alpha (Tnfα), and FIG. 20C) interleukin 17C (IL-17C) of dorsal skin tissues were suppressed after the treatment of pacDNA-1, as measured via quantitative real-time PCR (qRT-PCR) analysis. *p<0.05, **p<0.01 (two-tailed test).

FIGS. 21A-21B. 21. FIG. 21A) Structure of azide-modified B peptide. FIG. 21B) Mass spectrum of B pep-654 conjugate.

### DETAILED DESCRIPTION

A description of example embodiments follows.

### Abbreviations

ASO: antisense oligonucleotide
EGFP-654: modified enhanced green fluorescence protein (EGFP) pre-mRNA containing an aberrantly spliced human β-globin intron (IVS2-654)
IMQ: imiquimod
pacDNA: polymer-assisted compaction of DNA
Pi3: Serpina1c (official name: serine (or cysteine) peptidase inhibitor, clade A, member 1C)
PK: plasma pharmacokinetics
Tnfα: tumor necrosis factor alpha

### Definitions

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as some acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

### INTRODUCTION

The investigation of gene regulation therapeutics for the treatment of skin-related conditions is rarely explored in part due to inefficient systemic delivery. In this study, a bottlebrush polymer-antisense oligonucleotide (ASO) conjugate, termed pacDNA, designed to target, for example, IL-17 receptor A (IL-17RA), which is involved in psoriasis pathogenesis is presented. Systemic administration of pacDNA led to its accumulation in epidermis, dermis, and hypodermis of mouse skin, reduced IL-17RA gene expression in skin, and significantly reversed the development of imiquimod (IMQ)-induced psoriasis in a mouse model. These findings highlight the potential of the pacDNA as a promising nanoconstruct for systemic oligonucleotide delivery to the skin and for treating psoriasis and other skin-related conditions and disorders through systemic administration.

Examples of novel and/or unusual features of embodiments of the disclosed methods and compositions include high skin-site accumulation; the ability to engage with targets inside skin cells and skin-resident immune cells; and a non-toxic, non-immunogenic conjugate. Example advantages, improvements, problems solved by example embodiments include: pacDNA is able to deliver oligonucleotides to the skin in high efficiency. Using a psoriasis model, the pacDNA has significantly reduced the severity of the symptoms. The technology opens the door to new diseases currently without treatment. The technology provides a very high, if not highest, skin accumulation in preclinical models. In some embodiments, this can lead to reduced dosage or more effective drugs. Examples of potential uses for this invention include human or animal therapeutics, including those with indications that require skin delivery of oligonucleotides.

### Example Bottlebrush Polymer-Oligonucleotide Conjugates

In some embodiments of the disclosure, the compositions and methods provide for a method of treating a disease or disorder, comprising administering to a subject in need thereof, a therapeutically effective amount of a bottlebrush polymer-oligonucleotide conjugate. The terms “bottlebrush” and “brush” are used interchangeably herein. As used herein, the terms “polymer-assisted compaction of DNA” (pacDNA) and “BRUSHIELD®-DNA” or “Brushield™-DNA” refer to a bottlebrush polymer-oligonucleotide conjugate, wherein the oligonucleotide comprises a nucleic acid, e.g., DNA or RNA. As used herein, the term “BRUSHIELD®” or “Brushield™” is used to refer to the bottlebrush polymer portion of a bottlebrush polymer-oligonucleotide conjugate. In some instances, which will be clear to a person of skill in the art by context, “BRUSHIELD®)” or “Brushield™” may also be used to refer to BRUSHIELD®-DNA.

Embodiments of the bottlebrush polymer-oligonucleotide conjugate disclosed herein comprise a polymer backbone, polyethylene glycol (PEG) polymer arms (i.e., polymer side chains) covalently linked to the polymer backbone, and an oligonucleotide covalently linked to the backbone, wherein the oligonucleotide comprises a sequence complementary to a region of a transcript associated with a skin-related disease. In some embodiments, a bottlebrush polymer-oligonucleotide conjugate comprises a bottlebrush polymer, wherein the bottlebrush polymer comprises a polymer backbone and polyethylene glycol (PEG) polymer arms covalently linked to the polymer backbone.

In one aspect of the disclosure, there is provided a bottlebrush polymer-oligonucleotide conjugate comprising a polymer backbone, wherein the polymer backbone comprises a plurality of monomers. Each individual monomer can be covalently attached to an oligonucleotide or a polyethylene glycol (PEG) arm (i.e., one oligonucleotide per monomer, or one PEG arm per monomer). At least two of the plurality of monomers is covalently attached to a polyethylene glycol (PEG) arm (i.e., one PEG arm is attached to one monomer). In some embodiments, every monomer of the polymer backbone need not be covalently attached to an oligonucleotide or a polyethylene glycol (PEG) arm. In some embodiments, a monomer is attached to a PEG arm or derivatized with a functional group prior to being assembled into a polymer backbone. In some embodiments, a functionally derivatized monomer within a backbone is conjugated to an oligonucleotide.

The PEG arms and the at least one oligonucleotide can be attached to the polymer backbone in any order. For example, in some embodiments, a terminal monomer of the backbone is covalently linked to an oligonucleotide. In some embodiments, a terminal monomer of the backbone is covalently linked to a PEG arm. In some embodiments, an internal (e.g., non-terminal) monomer of the backbone is covalently linked to an oligonucleotide. In some embodiments, an internal monomer of the backbone is covalently linked to a PEG arm.

In some embodiments, a bottlebrush polymer-oligonucleotide conjugate comprises two or more oligonucleotides. In some embodiments, a bottlebrush polymer-oligonucleotide conjugate comprises three or more oligonucleotides. In some embodiments, a bottlebrush polymer-oligonucleotide conjugate comprises one oligonucleotide. In some embodiments, a bottlebrush polymer-oligonucleotide conjugate comprises two oligonucleotides. In some embodiments, a bottlebrush polymer-oligonucleotide conjugate comprises three oligonucleotides. In some embodiments, a bottlebrush polymer-oligonucleotide conjugate comprises one to about three oligonucleotides. In some embodiments, a bottlebrush polymer-oligonucleotide conjugate comprises two or more oligonucleotides, wherein each of the oligonucleotides comprises the same sequence. In some embodiments, a bottlebrush polymer-oligonucleotide conjugate comprises two or more oligonucleotides, wherein each of the oligonucleotides comprises different sequences.

In some embodiments, at least one of the monomers of the polymer backbone of a bottlebrush polymer-oligonucleotide conjugate is selected from a synthetic monomer, a natural monomer, and a modified form of a natural molecule. In some embodiments, at least one of the monomers is a synthetic monomer. In some embodiments, at least one of the monomers is a natural monomer. In some embodiments, at least one of the monomers is a modified form of a natural molecule. In some embodiments, at least one of the monomers is a synthetic monomer and at least one of the monomers is a natural monomer. In some embodiments, at least one of the monomers is a synthetic monomer and at least one of the monomers is a modified form of a natural molecule. In some embodiments, at least one of the monomers is a natural monomer and at least one of the monomers is a modified form of a natural molecule. In some embodiments, at least one of the monomers is a synthetic monomer, at least one of the monomers is a natural monomer, and at least one of the monomers is a modified form of a natural molecule. In some embodiments, a polymer backbone comprises a combination of one or more of a synthetic monomer, a natural monomer, and a modified form of a natural molecule.

In some embodiments, the synthetic monomer is selected from serinol, norbornene, acrylate, and acrylamide; the natural monomer is selected from amino acid and sugar; the modified form of a natural molecule is selected from a morpholino phosphorodiamidate, a modified amino acid, a modified spermine, a modified lipid, and a modified cholesterol; or any combination of the foregoing. In some embodiments, a synthetic monomer is selected from serinol, norbornene, acrylate, and acrylamide. In some embodiments, a synthetic monomer is serinol. In some embodiments, a synthetic monomer is norbornene. In some embodiments, a synthetic monomer is acrylate. In some embodiments, a synthetic monomer is acrylamide. In some embodiments, a natural monomer is selected from amino acid and sugar. In some embodiments, a natural monomer is an amino acid. In some embodiments, a natural monomer is a sugar. In some embodiments, a modified form of a natural molecule is selected from a morpholino phosphorodiamidate, a modified amino acid, a modified spermine, a modified lipid, and a modified cholesterol. In some embodiments, a modified form of a natural molecule is a morpholino phosphorodiamidate. In some embodiments, a modified form of a natural molecule is a modified amino acid. In some embodiments, a modified form of a natural molecule is a modified spermine. In some embodiments, a modified form of a natural molecule is a modified lipid. In some embodiments, a modified form of a natural molecule is a modified cholesterol.

In some embodiments, the backbone comprises one or more nanoparticles or monomers. In some embodiments, the backbone comprises one or more nanoparticles. An example of a nanoparticle is a fullerene C60. In some embodiments, the monomers are selected from synthetic monomers, natural monomers, or modified forms of natural molecules (e.g., natural monomers).

In some embodiments, a bottlebrush polymer-oligonucleotide conjugate comprises a diblock polymer, e.g., a copolymer comprising two different monomers. In some embodiments, a diblock polymer comprises two blocks, and each block comprising a different monomer. An example of two different monomers includes norbornenyl bromide (or a derivative thereof, e.g., norbornenyl azide or norbornenyl conjugated to an oligonucleotide (i.e., norbornenyl-oligonucleotide) and norbornenyl PEG. As used herein, the term “copolymer” means a polymer produced by addition polymerization between two or more different monomers. In some embodiments, a bottlebrush polymer-oligonucleotide conjugate comprises a triblock polymer, e.g., a copolymer comprising three different monomers, e.g., three blocks, wherein each block comprises a different monomer from an adjacent block.

In some embodiments, the bottlebrush polymer-oligonucleotide conjugate comprises about 15 to about 40 monomers. In some embodiments, the bottlebrush polymer-oligonucleotide conjugate comprises about 35 monomers. In some embodiments, the bottlebrush polymer-oligonucleotide conjugate comprises about 5-30 monomers. In some embodiments, the bottlebrush polymer-oligonucleotide conjugate comprises about 15 to about 30 norbornenyl PEG monomers. In some embodiments, the bottlebrush polymer-oligonucleotide conjugate comprises about 30 norbornenyl PEG monomers. In some embodiments, the bottlebrush polymer-oligonucleotide conjugate comprises about 30 norbornenyl PEG monomers and about 5 norbornenyl bromide monomers or derivatives of norbornenyl bromide monomers (e.g., norbornenyl azide or norbornenyl-oligonucleotide). In some embodiments, the bottlebrush polymer-oligonucleotide conjugate comprises about 30 norbornenyl PEG monomers and about 5 derivatives of norbornenyl bromide monomers (e.g., about 2 norbornenyl azide monomers and about 3 norbornenyl-oligonucleotide monomers).

In some embodiments, the bottlebrush polymer-oligonucleotide conjugate has a high molecular weight. In some embodiments, the bottlebrush polymer-oligonucleotide conjugate has a molecular weight of about 300 kDa.

In some embodiments, the bottlebrush polymer-oligonucleotide conjugate comprises about 15 to about 30 PEG polymer arms; each of the PEG polymer arms is about 2 kDa to about 20 kDa; or both of the foregoing. In some embodiments, the bottlebrush polymer-oligonucleotide conjugate comprises about 15 to about 30 PEG polymer arms. In some embodiments, the bottlebrush polymer-oligonucleotide conjugate comprises about 15 PEG polymer arms. In some embodiments, the bottlebrush polymer-oligonucleotide conjugate comprises about 30 PEG polymer arms. In some embodiments, each of the PEG polymer arms is about 2 kDa to about 20 kDa.

In some embodiments, the bottlebrush polymer-oligonucleotide conjugate comprises about 30 PEG polymer arms; each of the PEG polymer arms is about 10 kDa; or both of the foregoing. In some embodiments, the bottlebrush polymer-oligonucleotide conjugate comprises about 30 PEG polymer arms. In some embodiments, each of the PEG polymer arms is about 10 kDa.

In some embodiments, each of the PEG polymer arms comprises 10 kDa PEG (i.e., PEG with a molecular weight of about 10 kDa). In some embodiments, each of the PEG polymer arms comprises about 226 ethylene glycol units.

In some embodiments, the bottlebrush polymer-oligonucleotide conjugate exhibits a spherical morphology.

In some embodiments, the bottlebrush polymer-oligonucleotide conjugate further comprises a radiochemical label, a targeting ligand, or both. In some embodiments, the bottlebrush polymer-oligonucleotide conjugate further comprises a radiochemical label. In some embodiments, the bottlebrush polymer-oligonucleotide conjugate further comprises a targeting ligand. In some embodiments, the bottlebrush polymer-oligonucleotide conjugate further comprises a radiochemical label and a targeting ligand.

In another aspect, the disclosure provides a method of producing a bottlebrush polymer-oligonucleotide conjugate, comprising: synthesizing a bottlebrush polymer; and conjugating the bottlebrush polymer with an oligonucleotide comprising a sequence complementary to a region of a transcript associated with a skin-related condition, thereby producing the bottlebrush polymer-oligonucleotide conjugate.

In some embodiments, synthesizing the bottlebrush polymer comprises polymerizing norbornenyl bromide and norbornenyl PEG; and conjugating the bottlebrush polymer with the oligonucleotide comprises: conjugating the bottlebrush polymer with an azide group, thereby producing an azide-functionalized bottlebrush polymer; and reacting the azide-functionalized bottlebrush polymer and the oligonucleotide, wherein the oligonucleotide is a DBCO-modified oligonucleotide, thereby producing the bottlebrush polymer-oligonucleotide conjugate.

In another aspect, the disclosure provides a method of producing a bottlebrush polymer-oligonucleotide conjugate that comprises synthesizing a bottlebrush polymer; and conjugating the bottlebrush polymer with an ASO, thereby producing the bottlebrush polymer-oligonucleotide conjugate. In some embodiments, synthesizing the bottlebrush polymer comprises polymerizing norbornenyl bromide and norbornenyl PEG. In some embodiments, conjugating the bottlebrush polymer with an ASO comprises conjugating the bottlebrush polymer with an azide group, thereby producing an azide-functionalized bottlebrush polymer; and reacting the azide-functionalized bottlebrush polymer and the ASO, wherein the ASO is a DBCO-modified ASO.

### Example Oligonucleotides

In some embodiments, an oligonucleotide comprises a chemically modified double stranded nucleic acid. In some embodiments, an oligonucleotide (i.e., the oligonucleotide of a bottlebrush polymer-oligonucleotide conjugate) comprises a single stranded nucleic acid, a double stranded nucleic acid, a chemically modified nucleic acid, or any combination of the foregoing. In some embodiments, an oligonucleotide comprises a single stranded nucleic acid. In some embodiments, an oligonucleotide comprises a double stranded nucleic acid. In some embodiments, an oligonucleotide comprises a chemically modified nucleic acid.

In some embodiments, an oligonucleotide (i.e., the oligonucleotide of a bottlebrush polymer-oligonucleotide conjugate) has a length of about 8 bases or base pairs to about 35 bases or base pairs. In some embodiments, an oligonucleotide has a length of about 9 bases or base pairs, about 26 bases or base pairs, or about 31 bases or base pairs. In some embodiments, an oligonucleotide has a length of about 9 bases or base pairs. In some embodiments, an oligonucleotide has a length of less than about 50 bases or base pairs, for example, less than about 35 bases or base pairs. In some embodiments, an oligonucleotide has a length of more than about 8 bases or base pairs.

In some embodiments, the oligonucleotide comprises a single stranded nucleic acid or a double stranded nucleic acid. In some embodiments, the oligonucleotide comprises DNA bases, RNA bases, or a combination thereof.

In some embodiments, an oligonucleotide comprises an antisense oligonucleotide (ASO), a small interfering RNA (siRNA), or a splice-switching oligonucleotide (SSO). In some embodiments, an oligonucleotide comprises a sequence complementary to a region of a pathogenic transcript, e.g., an aberrant splice junction in an aberrantly spliced mRNA.

In some embodiments, an oligonucleotide comprises a small interfering RNA (siRNA). In some embodiments, an oligonucleotide comprises an antisense oligonucleotide (ASO). In some embodiments, the ASO comprises single stranded DNA or single stranded RNA; a chemically modified ASO or an unmodified ASO; or a combination of the foregoing. In some embodiments, the ASO comprises single stranded DNA or single stranded RNA. In some embodiments, the ASO comprises single stranded DNA. In some embodiments, the ASO comprises single stranded RNA. In some embodiments, the ASO comprises a chemically modified ASO or an unmodified ASO. In some embodiments, the ASO comprises unmodified ASO. In some embodiments, the ASO comprises locked nucleic acid (LNA) modified bases. In some embodiments, the ASO comprises a chemically modified ASO.

In some embodiments, an oligonucleotide comprises a chemically modified nucleic acid. In some embodiments, an oligonucleotide comprises one or more locked nucleic acid (LNA) modified bases. In some embodiments, an oligonucleotide comprises all locked nucleic acid (LNA) modified bases. In some embodiments, an oligonucleotide comprises one or more stabilizing modifications, functionalizing modifications, or both. As used herein the term “locked nucleic acid” (LNA), also known as bridged nucleic acid (BNA), is a synthetic nucleic acid analog wherein the ribose ring is conformationally locked by a methylene bridge connecting the 2′-O to the 4′-C.

Examples of chemical modifications that can be applied to oligonucleotides (e.g., ASOs) to produce chemically modified oligonucleotides include, but are not limited to: 2′ to 4′ methylene bridges (i.e., locked nucleic acid (LNA)); functionalization with DBCO (5′-Dimethoxytrityl-5-[(6-oxo-6-(dibenzo[b,f]azacyclooct-4-yn-1-yl)-capramido-N-hex-6-yl)-3-acrylimido]-2′-deoxyUridine,3′-[(2-cyanocthyl)-(N,N-diisopropyl)]-phosphoramidite)); a fluorescent dye (e.g., cyanine 3 (Cy3) or cyanine 5 (Cy5)); 2′-O-methylation; 2′-O-alkyl modifications; phosphorodiamidate morpholino oligomer (PMO); phosphorothioate (PS) linkage chemistry; and 4′-thio modifications. The aforementioned modifications and other modifications are well-known to those of skill in the art.

In some embodiments, an oligonucleotide of a bottlebrush polymer-oligonucleotide conjugate is designed to modulate gene expression, for example, by one or more of the following mechanisms: mRNA degradation, translation inhibition, splice modulation, RNA editing, or gene activation via RNAa.

In some embodiments, an oligonucleotide comprises a sequence complementary to a region of a transcript or a gene, e.g., a pathogenic transcript or a mutated gene. A region of a pathogenic transcript may be, for example, an aberrant splice junction, i.e., the site of an aberrant splicing event. As expressed herein, an oligonucleotide comprising a sequence complementary to a region of a pathogenic transcript is understood to “target” the pathogenic transcript. The term “pathogenic” refers to an agent that causes a disease or that is likely or suspected to cause a disease.

In some embodiments, the oligonucleotide comprises a sequence complementary to a region of a skin-related condition-associated transcript, gene, or both (see, e.g., Table 1). In some embodiments, the skin-related condition-associated transcript or gene comprises a human transcript or gene selected from: interleukin-17A (IL-17A), interleukin-17A receptor (IL-17RA), interleukin-23 (IL-23), interleukin-17F (IL-17F), interleukin-17C receptor (IL-17RC), tumor necrosis factor-alpha (TNF-α, i.e., TNFA), interferon-gamma (IFN-γ, i.e., IFNG), Janus kinase 1 (JAKI), Janus kinase 3 (JAK3), signal transducer and activator of transcription 3 (STAT3), tyrosinase (TYR), microphthalmia-associated transcription factor (MITF), collagen type I alpha 1 (COL1A1), matrix metalloproteinase 9 (MMP9), cyclin dependent kinase inhibitor 2A (CDKN2A); B-Raf proto-oncogene, serine/threonine kinase (BRAF); or any combination thereof.

TABLE 1
Example transcripts associated with skin-related conditions.

| Gene | | Primary Disease | Example |
|---|---|---|---|
| Target | Full Name | Category | Indications |
| IL-17A | Interleukin 17A | Inflammatory/ | Psoriasis, atopic |
| | | Autoimmune | dermatitis |
| IL-17RA | Interleukin 17 Receptor A | Inflammatory/ | Psoriasis, |
| | | Autoimmune | hidradenitis |
| | | | suppurativa |
| IL-23 | Interleukin 23 (p19) | Inflammatory/ | Psoriasis, lichen |
| | | Autoimmune | planus |
| IL-17F | Interleukin 17F | Inflammatory/ | Psoriasis |
| | | Autoimmune | |
| IL-17RC | Interleukin 17 Receptor C | Inflammatory/ | Psoriasis, eczema |
| | | Autoimmune | |
| TNF-α | Tumor Necrosis Factor Alpha | Inflammatory/ | Psoriasis, |
| | | Autoimmune | cutaneous lupus, |
| | | | hidradenitis |
| IFN-γ | Interferon Gamma | Autoimmune/ | Vitiligo, alopecia |
| | | Pigmentary | areata |
| JAK1 | Janus Kinase 1 | Inflammatory/ | Atopic dermatitis, |
| | | Autoimmune | alopecia areata |
| JAK3 | Janus Kinase 3 | Inflammatory/ | Atopic dermatitis, |
| | | Autoimmune | vitiligo |
| STAT3 | Signal Transducer and | Inflammatory/ | Psoriasis, |
| | Activator of Transcription 3 | Cancer | cutaneous |
| | | | squamous cell |
| | | | carcinoma |
| TYR | Tyrosinase | Pigmentary | Melasma, vitiligo, |
| | | Disorders | post-inflammatory |
| | | | hyperpigmentation |
| MITF | Microphthalmia-associated | Pigmentary/ | Melanoma, melasma |
| | Transcription Factor | Melanoma | |
| COL1A1 | Collagen Type I Alpha 1 Chain | Scarring/Wound | Keloids, |
| | | Healing | hypertrophic |
| | | | scars, ulcers |
| MMP9 | Matrix Metalloproteinase 9 | Wound Healing/ | Chronic wounds, |
| | | Scarring | burns, fibrosis |
| CDKN2A | Cyclin-Dependent Kinase | Cancer | Actinic keratosis, |
| (p16) | Inhibitor 2A | (Precancerous | melanoma |
| | | Lesions) | |
| BRAF | B-Raf Proto-Oncogene, | Cancer | Melanoma |
| | Serine/Threonine Kinase | | (especially V600E |
| | | | mutation) |

In some embodiments, the oligonucleotide comprises a sequence selected from SEQ ID NO: 1 through SEQ ID NO: 10 (Table 2). In some embodiments, the oligonucleotide targets a skin-related condition-associated transcript, gene, or both. In some embodiments, the bottlebrush polymer-oligonucleotide conjugate targets a skin-related condition-associated transcript, gene, or both.

TABLE 2
Example oligonucleotides that modulate transcripts
associated with skin-related conditions.

| | | | SEQ |
|---|---|---|---|
| | | | ID |
| Target | mRNA Region | Antisense Sequence (5′-3′) | NO: |
| IL-17RA | Exon 3 | GTCTGAGCTTCCAGATGAGA | 1 |
| IL-17RA | Exon 6 | AGGAGCATCTTGAGGATGGA | 2 |
| TYR | Exon 1 (start codon region) | CATGAGTGTGACTGCTGGTT | 3 |
| TYR | 3′ UTR | TGTTGATGGTCATGTCATGC | 4 |
| BRAF | Exon 15 (V600 region) | TGGATCCAGACAACTGTTCA | 5 |
| TNF-α | 5′ UTR | GAGTCCGGGCAGGTCTACTT | 6 |
| STAT3 | Exon 3 | CAGCCTGAGTTGTTGATGAT | 7 |
| MITF | Exon 2 | ATGTCCGTCATAGTGGTGGA | 8 |
| JAK1 | 3′ UTR | GGAGCTTTGGCTGTGTACTA | 9 |
| COL1A1 | Exon 49 (frequent dermal | GCTGAAGGACCTTGTTGTTT | 10 |
| | fibrosis hotspot) | | |

In some embodiments, the disclosure provides for compositions and methods of modulating or altering the expression of a gene product encoded by a target polynucleotide (e.g., IL-17RA mRNA). In some embodiments, the target polynucleotide is a polynucleotide specific to a mammalian cell (e.g., a mammalian cancer cell, a mammalian non-cancer cell, a mammalian muscle cell). In some embodiments, administration to the subject occurs in the absence of a transfection agent. As used herein, “transfection agent” refers to a means of crossing a cell membrane and/or nuclear envelope, wherein the means is not the bottlebrush polymer-oligonucleotide conjugate (i.e., the bottlebrush polymer-oligonucleotide conjugate does not comprise the transfection agent). In some embodiments a transfection agent is a transfection vector or a transfection reagent. In some embodiments, efficacy of administration is determined by measuring the subject's plasma pharmacokinetics, blood availability, extrahepatic distribution, tissue retention, dosing frequency or amount, or a combination thereof.

### Example Skin-Related Conditions

As used herein, the term “condition” includes diseases, disorders, syndromes, injuries, and other aspects that interfere with a state of healthfulness or well-being. As used herein, the terms “condition,” “disease,” and “disorder” may be used interchangeably. A disease may refer to a pathophysiological response to internal (e.g., genetic) or external (e.g., environmental) factors, or combinations thereof. A disorder may include an impairment of a normal state of an organ, tissue, or system within an individual, such as an interruption or modification of vital functions or performance of the organ, tissue, or system. A disease or disorder may be manifested by distinguishing signs, symptoms, or both. A syndrome may refer to a group of signs, symptoms, or both, for which an underling cause (e.g., a disease or a disorder) may be unknown or multifactorial.

As used herein, the term “skin” refers to the epidermis, dermis, hypodermis, dermal adipocyte layer (i.e., subcutaneous fat), or any combination thereof, or any constituent part thereof, e.g., a hair follicle or related structures, a tissue-resident macrophage, an exocrine gland of the skin (e.g., a sebaceous gland, an eccrine sweat gland, an apocrine sweat gland), and the like.

Examples of skin-related conditions include, for example: genetic skin disorders, including epidermolysis bullosa (e.g., epidermolysis bullosa simplex (EBS), junctional epidermolysis bullosa (JEB), Kindler epidermolysis bullosa (KEB), and dystrophic epidermolysis bullosa (DEB), e.g., recessive dystrophic epidermolysis bullosa (RDEB)), pachyonychia congenita, ichthyosis, Darier disease, and Netherton syndrome; inflammatory skin disorders, including psoriasis, atopic dermatitis, lichen planus, and hidradenitis suppurativa; autoimmune skin diseases, including vitiligo, alopecia areata, and cutaneous lupus erythematosus; skin cancers or precancers, including melanoma, basal cell carcinoma, squamous cell carcinoma, and actinic keratosis; infectious skin diseases, including human papillomavirus-induced lesions, herpes simplex virus infection, and molluscum contagiosum; wound healing disorders, including diabetic ulcers, radiation dermatitis, and burn wounds; scarring disorders, including keloids and hypertrophic scars; and pigmentary disorders, including melasma and post-inflammatory hyperpigmentation; and the like. Additional examples of skin-related conditions are known to those of skill in the art, including, for example, inflammatory skin diseases described in Liu et al. (Ref. 31).

In some embodiments, a skin-related condition comprises an injury or damage to the skin. Examples of skin injuries and skin damage include, but are not limited to: cuts, lacerations, gashes, tears, puncture wounds, surgical wounds, penetrating wounds (e.g., wounds that penetrate beyond the subcutaneous fat, e.g., avulsion), burns, pressure injuries (e.g., pressure sores), scratches, scrapes, abrasions, friction burns, contusions, hematomas, blisters, frostbite, sunburn, ulcers, scars, and the like. Examples of skin injuries include open wounds, closed wounds, injury that disrupt at least the epidermis, and injuries that penetrate the skin at least to the subcutaneous fat layer. Examples of other skin conditions or aspects of the skin include, but are not limited to: pigmentation irregularities (e.g., melasma), skin texture that is rough, dry, or scaly, uneven skin tone, rhytids (i.e., fine lines and wrinkles), eczema, rosacea, and acne.

### Subjects

The terms “subject” and “patient” are used interchangeably herein. The term “patient” refers to a human, while the term “subject” may refer to a human or a non-human animal. As used herein, “subject” or “patient” includes humans, domestic animals, such as laboratory animals (e.g., dogs, monkeys, pigs, rats, mice, etc.), household pets (e.g., cats, dogs, rabbits, etc.) and livestock (e.g., chickens, pigs, cattle (e.g., a cow, bull, steer, or heifer), sheep, goats, horses, etc.), and non-domestic animals. In some embodiments, a subject is a mammal (e.g., a non-human mammal). In some embodiments, a subject is a human. In still further embodiments, a subject of the disclosure may be a cell, cell culture, tissue, organ, or organ system.

In some embodiments the subject is about 0-3 months, 0-6 months, 6-11 months, 12-15 months, 12-18 months, 19-23 months, 24 months, 1-2 years, 2-3 years, 4-6 years, 7-10 years, 11-12 years, 11-15 years, 16-18 years, 18-20 years, 20-25 years, 25-30 years, 30-35 years, 30-40 years, 35-40 years, 30-50 years, 30-60 years, 50-60 years, 60-70 years, 50-80 years, 70-80 years, 80-90 years, or older than 60 years of age.

In some embodiments, a subject has or is at risk of developing a skin-related condition. In some embodiments, a subject has or is at risk of having an aspect of the skin that may be improved.

### Example Methods of Treatment, Diagnosis, and Improving an Aspect of Skin

As used herein, “therapy,” “treat,” “treating,” or “treatment” means inhibiting or relieving a condition in a subject in need thereof. For example, a therapy or treatment refers to any of: (i) the prevention of symptoms associated with a disease or disorder (e.g., psoriasis); (ii) the postponement of development of the symptoms associated with a disease or disorder (e.g., psoriasis); and/or (iii) the reduction in the severity of such symptoms that will, or are expected, to develop with said disease or disorder (e.g., psoriasis). In some embodiments, a therapy or treatment is useful in the diagnosis of a condition or an improvement of a condition or aspect of the skin. The terms include ameliorating or managing existing symptoms, preventing additional symptoms, and ameliorating or preventing the underlying causes of such symptoms. Thus, the terms denote that a beneficial result is being conferred on at least some of the subjects (e.g., humans) being treated. Many therapies or treatments are effective for some, but not all, subjects that undergo the therapy or treatment. In some embodiments, the symptoms comprise one or more of the symptoms described herein.

As used herein, the term “effective amount” means an amount of a composition, that when administered alone or in combination to a cell, tissue, or subject, is effective to achieve the desired therapy or treatment under the conditions of administration. For example, an effective amount is one that would be sufficient to produce an immune response to bring about effectiveness of a therapy (therapeutically effective) or treatment. The effectiveness of a therapy or treatment (e.g., eliciting a humoral and/or cellular immune response) can be determined by suitable methods known in the art.

In some embodiments, the conjugate requires reduced dosing and dosing frequency for oligonucleotide drugs. In some embodiments, the conjugate exhibits increased potency compared to other therapies for skin-related conditions.

In some embodiments, administering a bottlebrush polymer-oligonucleotide conjugate to a subject induces alternative splicing. In some embodiments, administering a bottlebrush polymer-oligonucleotide conjugate to a subject induces exon skipping. In some embodiments, an effect of administering a bottlebrush polymer-oligonucleotide conjugate to a subject is determined by the nucleic acid sequence of the oligonucleotide.

In some embodiments, administering the bottlebrush polymer-oligonucleotide conjugate to the subject induces splicing correction. In some embodiments, administering the bottlebrush polymer-oligonucleotide conjugate to the subject induces global splicing correction. As used herein, “splicing correction” refers to reverting an aberrant splicing (mis-splicing) pattern or a pathogenic splicing pattern to a wild-type splicing pattern or a non-pathogenic splicing pattern.

In some embodiments, the composition is administered to the subject as a dose of about 0.5 mg oligonucleotide per kg bodyweight (0.5 mg/kg) to about 5 mg oligonucleotide per kg bodyweight (5 mg/kg); by a systemic injection, for example, intravenous injection, intramuscular injection, intraperitoneal injection, or subcutaneous injection; or any combination of the foregoing.

In some embodiments, a composition comprising a bottlebrush polymer-oligonucleotide conjugate is administered to the subject: once a week for twelve consecutive weeks; once a day for four consecutive days, then once a week for twelve consecutive weeks;

once a month for at least two consecutive months; or once every two to three months for at least two consecutive cycles. In some embodiments, a composition may be dosed intravenously (i.v.) every one month, two months or three months.

In some embodiments, the composition is administered to the subject by injection, aerosol inhalation, infusion, ingestion, or a combination thereof. In some embodiments, the composition is administered to the subject by injection. In some embodiments, the composition is administered to the subject by intravenous injection, intramuscular injection, or subcutaneous injection. In some embodiments, the composition is administered to the subject by intravenous (i.v.) injection.

In some embodiments, the composition is administered to the subject as a topical formulation, for example, a cream, a hydrogel, a patch, a microneedle array, or a spray. In some embodiments, a topical formulation comprises one or more excipients, diluents, and carriers that facilitate bottlebrush polymer-oligonucleotide conjugate absorption into the epidermis. In some embodiments, a topical formulation comprising the bottlebrush polymer-oligonucleotide conjugate comprises about 0.2 to 10 μM oligonucleotide. In some embodiments, a topical formulation may be administered to the subject, or may be self-administered by the subject, between once a week and daily, more than once a day, or pro re nata. In some embodiments, the composition is administered to the subject topically, intradermally, or transdermally at an appropriate dose, e.g., measured as mg oligonucleotide per cm² of skin.

In some embodiments, a bottlebrush polymer-oligonucleotide conjugate or a composition comprising the conjugate exhibits increased stability (e.g., half-life) compared to existing treatments.

Determining the dosage and route of administration for a particular agent, patient and disease or condition is well within the abilities of one of skill in the art. In certain embodiments, the administration of the composition may be carried out in any manner, e.g., by parenteral or nonparenteral administration, including by aerosol inhalation, injection, infusions, ingestion, transfusion, implantation or transplantation. For example, the compositions described herein may be administered to a patient trans-arterially, intradermally, subcutaneously, intratumorally, intramedullary, intranodally, intramuscularly, by intravenous (i.v.) injection, intranasally, intrathecally or intraperitoneally. In one aspect, the compositions of the present disclosure are administered intravenously. In one aspect, the compositions of the present disclosure are administered to a subject by intramuscular or subcutaneous injection. The compositions may be injected, for instance, directly into a tumor, lymph node, tissue, organ, or site of infection. In some embodiments the route of administration is intramuscular, intranodal, intravenous, intradermal, subcutaneous, intranasal, infusion, intraperitoneal, intracranial, intratracheal or epicardial. Preferably, the dosage does not cause or produces minimal adverse side effects. In some embodiments, the conjugate is administered with phosphate-buffered saline (PBS).

In some embodiments, the route of administration is determined by the tissue or tissues, or organ to which the agent or agents are targeted. In some embodiments, the tissue, tissues, or organ is the subject's lung, ovary, immune system, skin, blood vessel, muscle, blood, brain, heart, intestine(s), pancreas, spleen, kidney, heart, bone, bone marrow, stomach, head, or any combination thereof.

In another aspect, the disclosure provides a composition that comprises a bottlebrush polymer-oligonucleotide conjugate. In some embodiments, the compositions and methods further comprise a pharmaceutically acceptable carrier. As used herein, the term “pharmaceutically acceptable” refers to those which are, within the scope of sound medical judgment, suitable for use without undue toxicity, irritation, allergic response and the like, and are commensurate with a reasonable and appropriate benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

In some embodiments, compositions as described herein are used in combination with other known agents (e.g., additional therapeutic agents) and therapies, which are used for treatment of skin related disorders, e.g., psoriasis. As used herein, “combination” may refer to, for example, contemporaneous treatment of a subject with a disclosed conjugate and with another agent, or to treatment of a subject with a composition comprising both a disclosed conjugate and another agent. In some embodiments, compositions as described herein are used in combination with other known agents (e.g., additional therapeutic agents) and therapies, such as chemotherapy, transplantation, and radiotherapy. Administered “in combination”, as used herein, means that two (or more) different treatments are delivered to the subject during the course of the subject's treatment e.g., the two or more treatments are delivered after the subject has been diagnosed with the condition and before the condition has been cured or eliminated or treatment has ceased for other reasons. In some embodiments, different treatments (e.g., additional therapeutics) can be administered separately, simultaneously or sequentially. Examples of additional therapeutics that may be administered to treat, for example, psoriasis, are described by Rendon and Schäkel (Ref. 32). In some embodiments, an additional therapeutic comprises an antibody or an antigen-binding fragment thereof. In some embodiments, an antibody is a bispecific antibody, i.e., an antibody that binds two distinct antigens or targets. In some embodiments, compositions comprise other known agents (e.g., additional therapeutic agents) which are used for treatment of skin related disorders, e.g., psoriasis.

### Additional Methods

In some aspects, the disclosure provides a method of delivering a therapeutic oligonucleotide to the skin of a subject in need thereof, wherein the method comprises administering to the subject a composition comprising a bottlebrush polymer-oligonucleotide conjugate, wherein the conjugate comprises: a polymer backbone, polyethylene glycol (PEG) polymer arms covalently linked to the polymer backbone, and an oligonucleotide covalently linked to the backbone. In some embodiments, the oligonucleotide modulates expression of one or more transcripts associated with a skin-related condition.

In some aspects, the disclosure provides a method of modulating expression of one or more transcripts associated with a skin-related condition in the skin of a subject in need thereof, the method comprising administering to the subject a composition comprising a bottlebrush polymer-oligonucleotide conjugate, wherein the conjugate comprises: a polymer backbone, polyethylene glycol (PEG) polymer arms covalently linked to the polymer backbone, and an oligonucleotide covalently linked to the backbone. In some embodiments, the oligonucleotide modulates expression of one or more transcripts associated with a skin-related condition.

In some embodiments, the oligonucleotide comprises a sequence complementary to a gene target listed in Table 1, i.e., targets a gene listed in Table 1. In some embodiments, an oligonucleotide that targets a gene may be used in a method of treating, preventing, or diagnosing a skin-related disease category or indication corresponding to that gene target, for example, as shown in Table 1. For example, in some embodiments, the oligonucleotide targets IL-17A and is used in a method of treating, preventing, or diagnosing an inflammatory and/or autoimmune disease, e.g., psoriasis or atopic dermatitis.

### Example Pharmaceutical Compositions

In some aspects, a composition, e.g., a pharmaceutical composition comprising a bottlebrush polymer-oligonucleotide conjugate, is provided herein.

The term “pharmaceutically acceptable salts” embraces salts commonly used to form alkali metal salts and to form addition salts of free acids or free bases. The nature of the salt is not critical, provided that it is pharmaceutically acceptable.

Suitable pharmaceutically acceptable acid addition salts may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts include, but are not limited to, metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. Pharmaceutically acceptable basic/cationic salts also include, the diethanolamine, ammonium, ethanolamine, piperazine and triethanolamine salts.

All of these salts may be prepared by conventional means by treating, for example, a composition described herein with an appropriate acid or base.

A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in subjects.

In some embodiments, the pharmaceutical composition is formulated as a solution. In some embodiments, a pharmaceutical composition suitable for use in methods of the disclosure further comprises one or more pharmaceutically acceptable carriers.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., oligonucleotide) is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton; sodium octyl glycoside; lauryl-, myristyl-, linolcyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linolcamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

Agents (e.g., oligonucleotide) disclosed herein may be prepared in accordance with standard procedures and are administered at dosages that are selected to reduce, prevent, or eliminate, or to slow or halt progression of, a condition being treated (See, e.g., Remington's Pharmaceutical Sciences, Mack Publishing Company, Easton, PA, and Goodman and Gilman's The Pharmaceutical Basis of Therapeutics, McGraw-Hill, New York, N. Y., the contents of which are incorporated herein by reference, for a general description of methods for administering various agents for human therapy).

In some embodiments, compositions of the disclosure are administered in a delivery vehicle comprising a nanocarrier selected from the group consisting of a lipid, a polymer and a lipo-polymeric hybrid. In still further embodiments, the first and second polynucleotides are encapsulated in a lipid nanoparticle, polymer nanoparticle, virus-like particle, nanowire, exosome, or hybrid lipid/polymer nanoparticle. In some embodiments, the first and second polynucleotides are encapsulated in the same nanocarrier. In still further embodiments, the first and second polynucleotides are encapsulated in different nanocarriers. In some embodiments, the lipid nanoparticle is ionizable.

As used herein, the term “pharmaceutically acceptable” refers to species which are, within the scope of sound medical judgment, suitable for use without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

In some embodiments, the composition is administered to the subject once a week for twelve consecutive weeks. In some embodiments, the composition is administered to the subject once a day for four consecutive days, then once a week for twelve consecutive weeks. In some embodiments, the composition is administered to the subject once a month for at least two consecutive months. In some embodiments, the composition is administered to the subject once every two to three months for at least two consecutive cycles.

A desired dose may conveniently be administered in a single dose, for example, such that the agent is administered once per day, or as multiple doses administered at appropriate intervals, for example, such that the agent is administered 2, 3, 4, 5, 6 or more times per day. The daily dose can be divided, especially when relatively large amounts are administered, or as deemed appropriate, into several, for example 2, 3, 4, 5, 6 or more, administrations. Typically, the compositions will be administered from about 1 to about 6 (e.g., 1, 2, 3, 4, 5 or 6) times per day or, alternatively, as an infusion (e.g., a continuous infusion). In some embodiments, the administration of the bottlebrush polymer-oligonucleotide conjugate may be repeated after one day, two days, three days, four days, five days, six days, one week, two weeks, three weeks, four weeks, five weeks, six weeks, seven weeks, two months, three months, four months, five months, six months or longer. The repeated administration may be at the same dose or at a different dose.

Doses lower or higher than those recited above may be required. Specific dosage and treatment regimens for any particular subject will depend upon a variety of factors, for example, the activity of the specific agent employed, the age, body weight, general health status, sex, diet, time of administration, rate of excretion, drug combination, the severity and course of the disease, condition or symptoms, the subject's disposition to the disease, condition or symptoms, the judgment of the treating physician and the severity of the particular disease being treated. The amount of an agent in a composition will also depend upon the particular agent in the composition.

EXEMPLIFICATION

To date, the development of oligonucleotides therapeutics has been concentrated on a limited set of organs and tissues including the liver, skeletal muscle, and the central nervous system. While the skin is the largest vascularized organ of the body, the distribution and retention of intravenously (i.v.) injected oligonucleotide formulations in the skin is not as well studied (Ref. 1). The lack of interest in the skin may be in part due to the much lower content of blood circulating oligonucleotides absorbed in the skin compared to the kidney, liver, and spleen, as suggested by current studies (Ref. 2).

A form of PEGylated oligonucleotide, termed pacDNA (polymer-assisted compaction of DNA), which consists typically of 1-3 ASO strands covalently attached to the backbone of bottlebrush polymer with about 30 polyethylene glycol (PEG) side chains (Ref. 3). Such a structure, interestingly, does not generate anti-PEG antibodies after repeated i.v. dosing, while linear PEG under an identical dosing regimen would lead to significant adaptive immunity. In addition, pacDNA exhibits a favorable safety profile (Ref. 4). Being sufficiently large to evade renal clearance, the pacDNA exhibits markedly prolonged blood circulation times with two orders of magnitude greater plasma area-under-the curve compared to free oligonucleotides (Ref. 5). Without being bound by theory, the long plasma pharmacokinetics (PK) combined with the rich terminal networks of blood supply capillaries in the skin may provide the bottlebrush polymer a kinetic advantage to be absorbed by skin cells, which makes the pacDNA a promising vehicle to address skin diseases using the oligonucleotide modality.

The present disclosure reports an investigation of pacDNA in a psoriasis-like mouse model. Psoriasis is an immune-mediated chronic inflammatory skin disease characterized by keratinocytes hyperproliferation, parakeratosis, and immune cells infiltration (including monocytes, T-cells, and neutrophils) (Ref. 6). Recent studies demonstrate that psoriasis pathogenesis strongly depends on the interleukin-23/interleukin 17 (IL-23/IL-17) axis; the cytokines such as IL-17A and IL-17F which are secreted by T-helper 17 (Th17) cells play a critical role in the development of the disease (Ref. 7). Therefore, many biological agents targeting Th17 cells, their related cytokines, and receptors such as IL-17 receptor A (IL-17RA) have been developed to treat psoriasis (Ref. 8). For example, both systemic administration of monoclonal antibodies blocking IL-17A (ixekizumab, see Ref. 9; and secukinumab, see Ref. 10) and IL-17RA (brodalumab, see Ref. 11) have shown high efficacy for the treatment of moderate-to-severe psoriasis cases (Ref. 12). Nucleic acid based skin therapeutic candidates, including ASOs, microRNAs, and small interfering RNAs (siRNAs), have been envisioned but are largely focused on topical transdermal delivery, which suffer from poor skin penetration, short half-lives, and limited target engagement (Ref. 13). Systemic application of nucleic acid-based therapeutics to treat skin pathologies can in principle alleviate these difficulties but poses a significant delivery challenge (Ref. 14).

In summary, the Examples set forth below demonstrate that pacDNA is a highly effective agent to realize skin delivery of oligonucleotide therapeutics with systemic administration. The conjugate preferentially accumulates in the skin, distributes across all skin layers, and gains access to skin cells and skin-resident immune cells. The elevated skin uptake and retention allow a markedly lower dose of oligonucleotide to be used compared to conventional formulations to achieve a comparable phenotypic response. Using an imiquimod (IMQ)-induced psoriasis mouse model, the pacDNA effectively engages with the target, reduces the severity of the symptoms, and reverses the development of psoriasis phenotypically, translationally, and histologically. This study opens the door for new therapies for a range of skin disorders with a genetic basis that are previously recalcitrant to treatment.

### Example 1

Preparation and Characterization of pacDNA

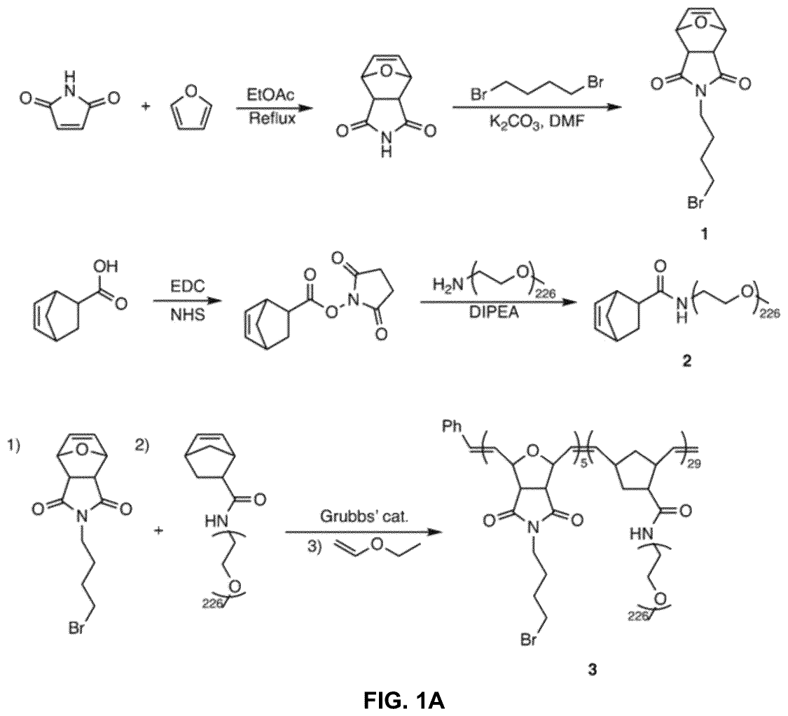

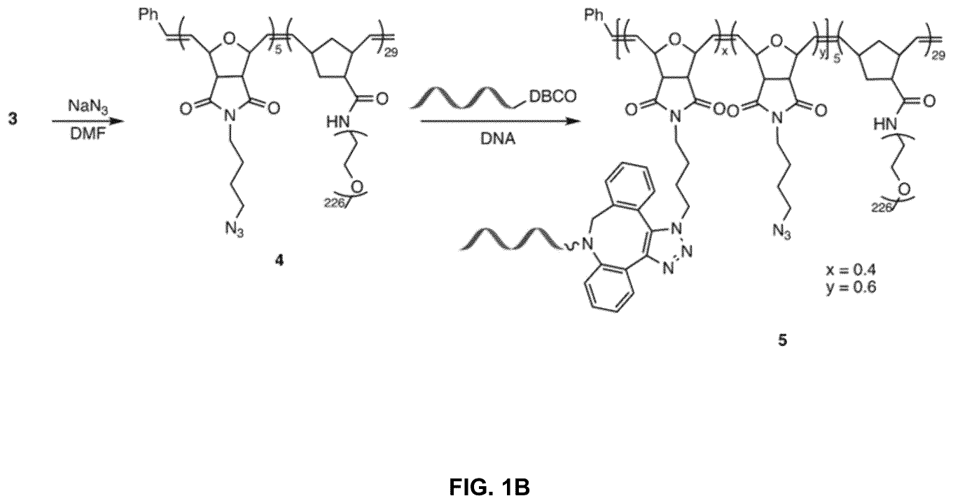

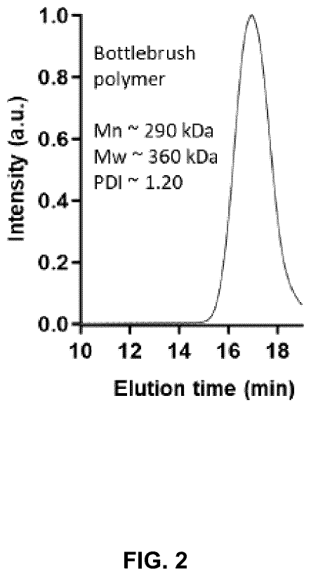

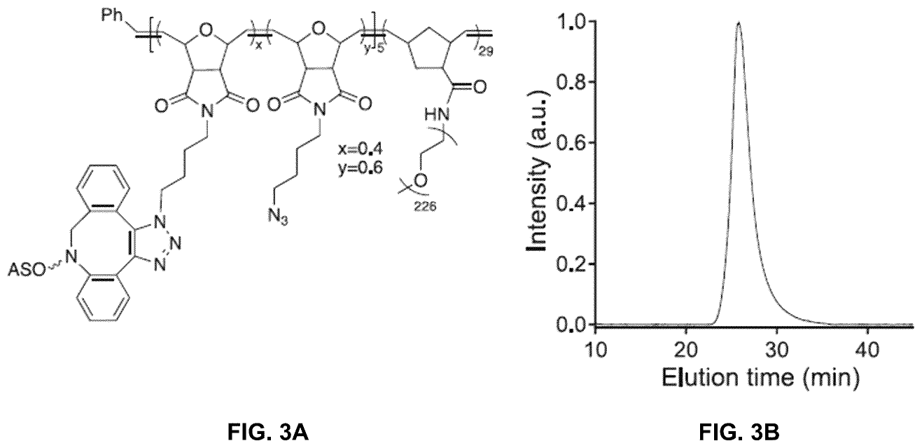

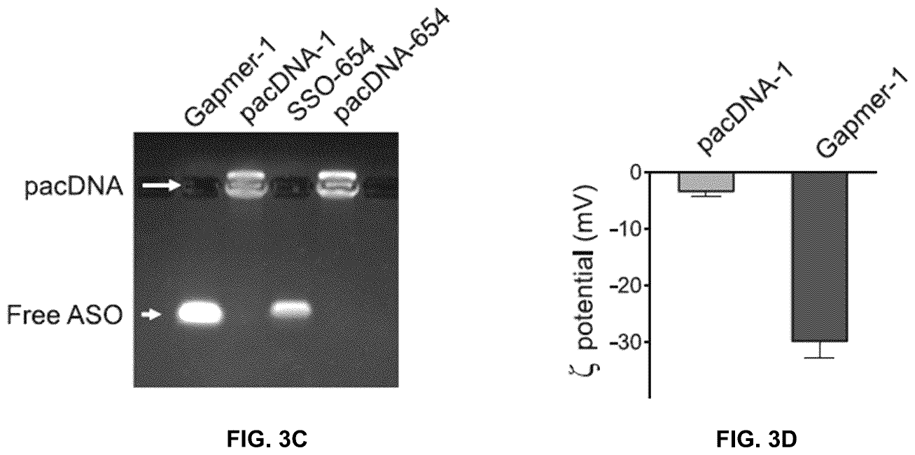

The synthesis of pacDNA followed protocols as described in prior literature (FIGS. 1A-1B) (Ref. 15). Tables 3-4 present the antisense oligonucleotide (ASO) sequences used in this study. Briefly, to synthesize the diblock bottlebrush polymer, a sequential ring-opening metathesis polymerization (ROMP) reaction is employed using two monomers: 7-oxanorbornenyl bromide (N-Br; see FIG. 1A, compound 1) (Ref. 16) and norbornenyl-modified PEG (N-PEG; compound 2) at a ratio of 5:35. The reaction yields a diblock architecture (pNBr₅-bpNPEG₂₉, yield: 93%, number average molar mass (Mn): 290 kDa, polydispersity index (PDI): 1.20; compound 3), which was characterized by N,N-dimethylformamide gel permeation chromatography (DMF-GPC) (FIG. 2). Next, azide substitution of bromides and subsequent coupling with dibenzocyclooctyne (DBCO)-modified ASO strands were carried out (see FIG. 1B, compounds 4 and 5). After the removal of unconjugated ASOs by aqueous GPC, purified pacDNA conjugates with an average of 1.9 ASO strands per polymer were obtained (FIGS. 3A-3D). Agarose gel electrophoresis showed significantly decreased electrophoretic mobility compared with free ASO following the conjugation reaction. Dynamic light scattering (DLS) revealed the presence of nanoparticles with a Z-average hydrodynamic diameter of 30±2 nm. Furthermore, and ζ potential measurements indicated that pacDNA-1, which contains a gapmer ASO (gapmer-1, Tables 3-4), exhibited a slight negative charge of about −4 to −2 millivolts (mV), which represents a significant reduction in negative surface charge compared to the free gapmer of about −30 mV.

TABLE 3
ASO sequences used in the study.

| SEQ ID | | |
|---|---|---|
| NO: | Name | Sequence |
| 11 | Cy5-labeled IL-17RA | 5′-AGTCATCACCATGTTTCT-Cy5-3′ |
| | antisense DNA | |
| 12 | Cy5-labeled IL-17RA | 5′-A*GUCATCACCATGTTUC*U-Cy5-3′ |
| | antisense gapmer | |
| | (gapmer-1) | |
| 12 | DBCO-modified IL- | 5′-DBCO-A*GUCATCACCATGTTUC*U-3′ |
| | 17RA antisense gapmer | |
| | (gapmer-1) | |
| 12 | Cy5-labeled and | 5′-DBCO-A*GUCATCACCATGTTUC*U-Cy5-3′ |
| | DBCO-modified IL- | |
| | 17RA antisense gapmer | |
| | (gapmer-1) | |
| 15 | DBCO modified | 5′-DBCO-G*UUTCACCACCCAATUC*C-3′ |
| | scramble gapmer | |
| | (gapmer-2) | |
| 16 | DBCO-modified SSO- | 5′-DBCO-GCTATTACCTTAACCCAG-3′ |
| | 654 | |

Unless otherwise noted: Phosphodiester (PO) DNA.
Asterisk (*): phosphorothioate internucleotide linkage.
Underline: RNA with 2′-O-methyl (OMe) modification.
Bold: locked nucleic acid (LNA) modification.

TABLE 4
Alternative notation of ASO sequences shown in Table 3.

| SEQ | | |
|---|---|---|
| ID | | |
| NO: | Name | 5′ Sequence |
| 11 | Cy5-labeled IL- | A^{P}G^{P}T^{P}C^{P}A^{P}T^{P}C^{P}A^{P}C^{P}C^{P}A^{P}T^{P}G^{P}T^{P}T^{P}T^{P}C^{P}T^{P}-Cy5 |
| | 17RA antisense | |
| | DNA | |
| 12 | Cy5-labeled IL- | A^{O}*G^{O}UºC^{P}A^{P}T^{P}C^{P}A^{P}C^{P}C^{P}A^{P}T^{P}G^{P}T^{P}T^{P}U^{O}C^{O}*U^{O}-Cy5 |
| | 17RA antisense | |
| | gapmer (gapmer-1) | |
| 12 | DBCO-modified IL- | DBCO-A^{O}*GOU^{O}C^{P}A^{P}T^{P}C^{P}A^{P}C^{P}C^{P}A^{P}T^{P}G^{P}T^{P}T^{P}U^{O}C^{O}*UO |
| | 17RA antisense | |
| | gapmer (gapmer-1) | |
| 12 | Cy5-labeled and | DBCO-A^{O}*GOU^{O}C^{P}A^{P}T^{P}C^{P}A^{P}C^{P}C^{P}A^{P}T^{P}G^{P}T^{P}T^{P}U^{O}C^{O}*U^{O}-Cy5 |
| | DBCO-modified IL- | |
| | 17RA antisense | |
| | gapmer (gapmer-1) | |
| 15 | DBCO modified | |
| | scramble gapmer | |
| | (gapmer-2) | |
| 16 | DBCO-modified | DBCO-G^{L}C^{L}T^{L}A^{L}T^{L}T^{L}A^{L}C^{L}C^{L}T^{L}T^{L}A^{L}A^{L}C^{L}C^{L}C^{L}A^{L}G^{L} |
| | SSO-654 | |

^{P}Phosphodiester (PO) DNA base without modifications; *phosphorothioate internucleotide linkage; ^{O}RNA with 2′-O-methyl (OMe) modification; ^{L}locked nucleic acid (LNA) modification.

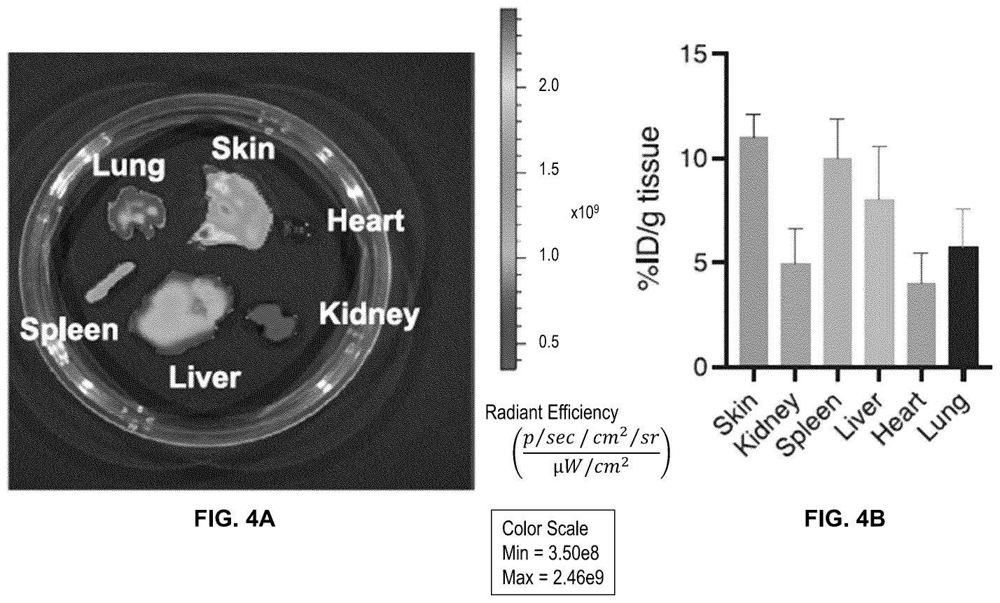

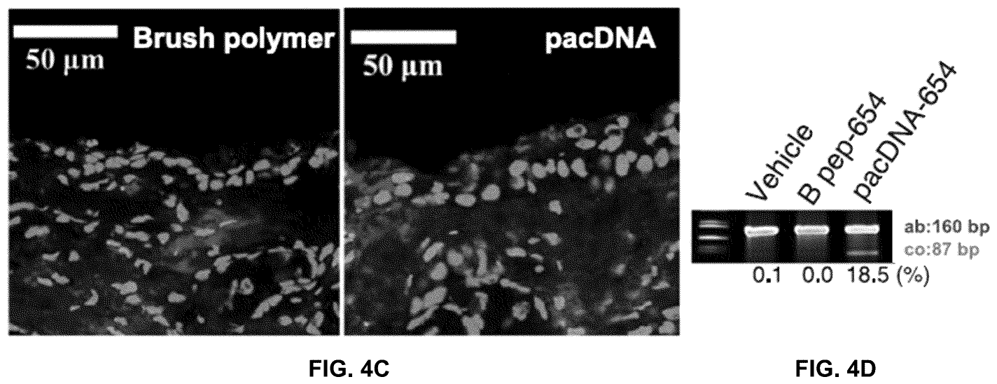

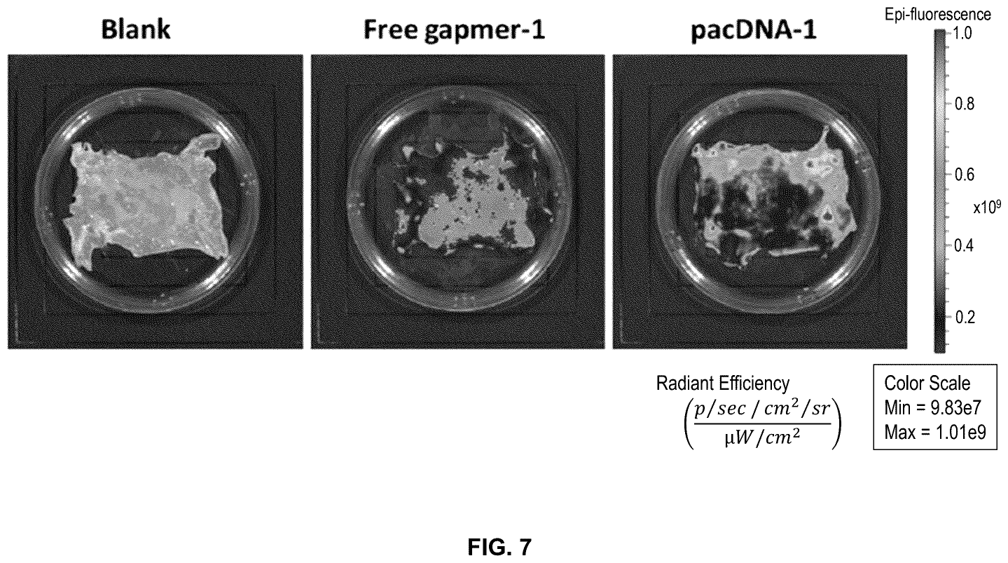

To assess the biodistribution of bottlebrush polymer, cyanine5 (Cy5)-labeled bottlebrush polymer was injected into the tail vein of C57BL/6 mice. Fluorescence imaging of dissected major internal organs and the skin 24 hours after injection was carried out, showing pronounced uptake into the mouse skin (FIG. 4A). Radiant efficiency (FIGS. 4A, 7) is calculated as photons (p) per second (sec) radiated from an area of tissue (cm²) into a solid angle of 1 steradian (sr): (p/see/cm²/sr)/(μW/cm²).

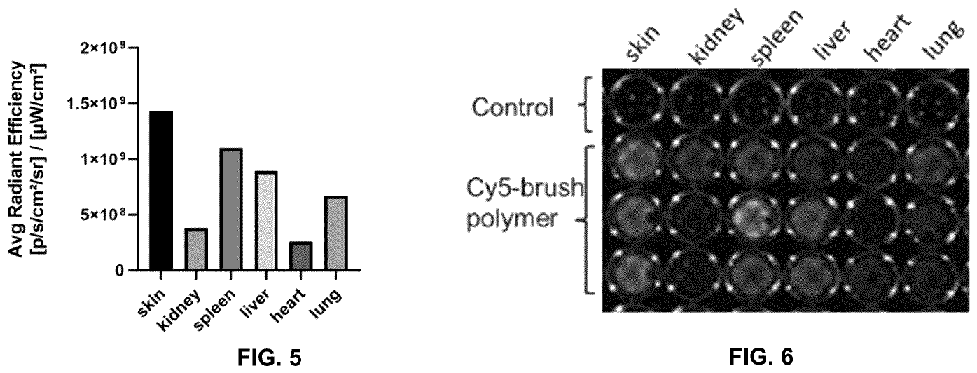

In fact, the skin exhibited the highest uptake level among all organs tested, as determined by average radiant efficiency (FIG. 5) or by fluorescence intensity of tissue homogenates (FIG. 4B; FIG. 6). The latter allows determination of the skin uptake as percent of the injected dose per gram (% ID/g) of skin tissue. The bottlebrush polymer exhibited 11.0% ID/g in the skin. For comparison, 2′-O-(2-methoxyethyl)-modified (OMe-modified) phosphorothioate (PS) oligonucleotide shows skin uptake of 0.17% ID/g (Ref. 17), while co-carriers considered of clinical relevance for oligonucleotide delivery such as cationic liposomes have the skin uptake at about 1.81% ID/g (Ref. 18). Of note, the comparison is approximate as dosing, dosing frequency, and species vary across different studies. This effect may be attributable to the extended plasma pharmacokinetics (PK) and the rich vasculature of skin tissues. The polymer is able to impart its skin distribution to conjugated oligonucleotides, as pacDNA-1's skin distribution is significantly more evident compared with the free gapmer-1 as determined by ex vivo imaging (FIG. 7).

### Example 2

### Skin Distribution

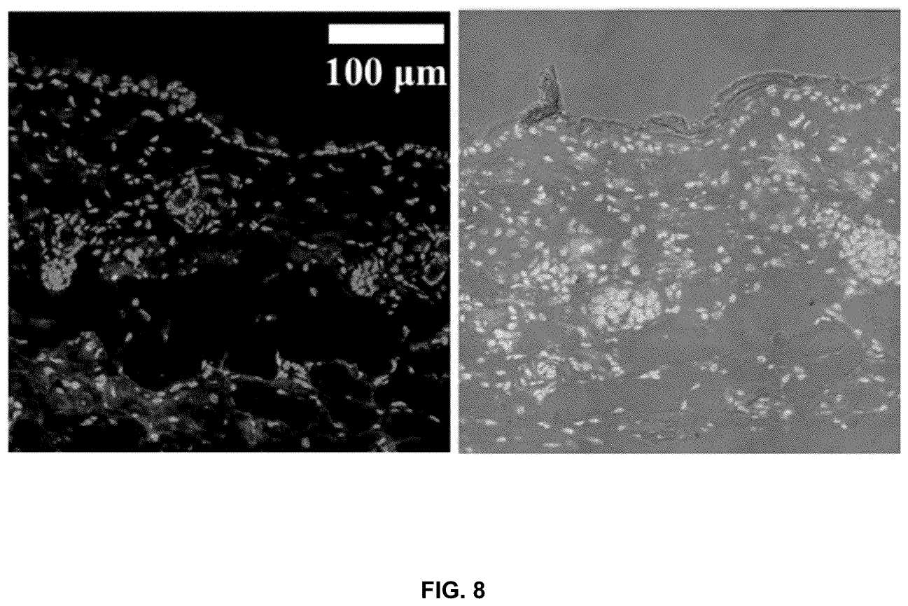

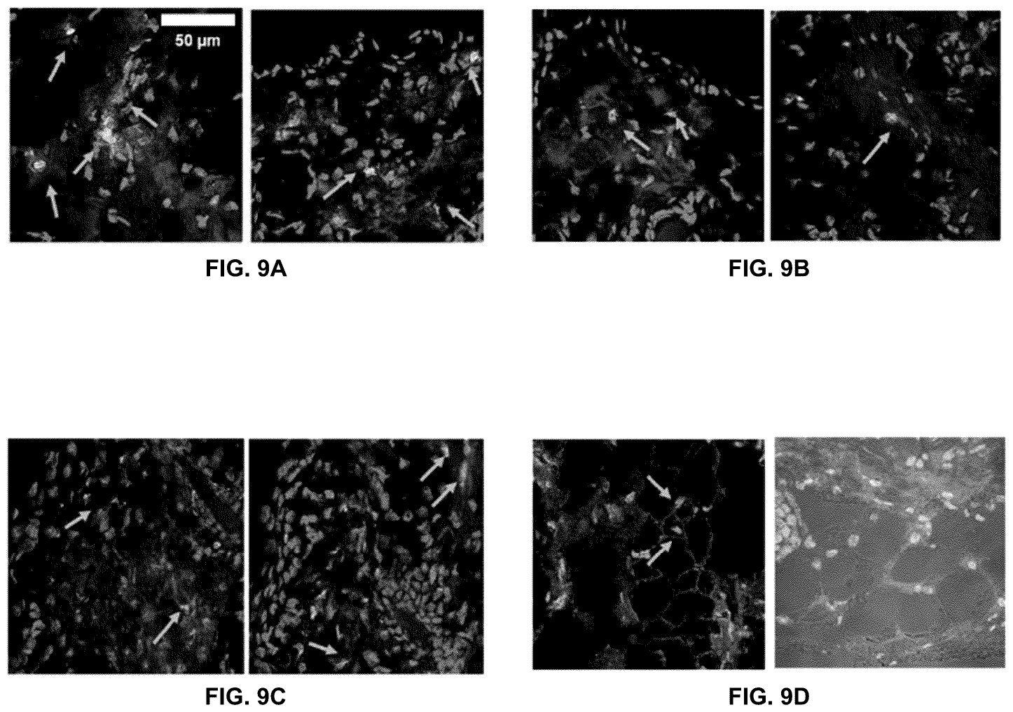

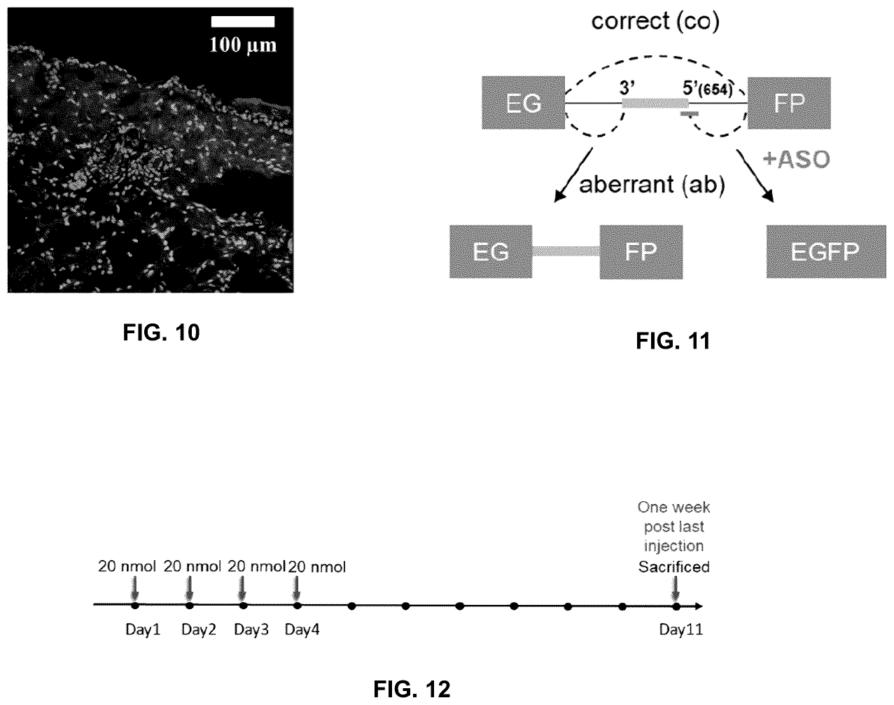

Next, an immunohistological study regarding the sub-organ localization of brush polymer within the skin was performed. Twenty-four hours after i.v. injection of Cy5-labeled bottlebrush polymer, the dorsal skin tissues of C57BL/6 mice were harvested, and fluorescence microscopy of cryosectioned skin revealed that the bottlebrush polymer was present across the epidermis, dermis, and hypodermis (FIG. 4C; FIG. 8). Immunofluorescence staining showed that the polymer was taken up by skin resident macrophages (F4/80+) (see Ref. 19), dendritic cells (CD11c+) (see Ref. 19), fibroblasts (vimentin) (see Ref. 20), and adipose cells (perilipin-1 and bright field) (Ref. 21) (FIG. 9). In addition, fluorescence signals were observed in pericellular space of the dermis which may be due to diffusion and exocytotic activities (Ref. 1b). When gapmer-1 was conjugated to the bottlebrush polymer (i.e., the pacDNA), a similar signal intensities and distribution pattern was observed compared to the bottlebrush polymer alone (FIG. 4C; FIG. 10).

### Example 3

### Splicing Switching in the Skin

Next, the antisense activity of i.v. injected pacDNA in the skin was evaluated utilizing the EGFP-654 transgenic mice, which express a modified enhanced green fluorescence protein (EGFP) pre-mRNA containing an aberrantly spliced human β-globin intron (IVS2-654) (Refs. 15, 22). When a splice-switching oligonucleotide (SSO) binds to the aberrant 5′ splicing site (FIG. 11), correct splicing can be restored, resulting in the subsequent production of functional EGFP mRNA (Ref. 23). EGFP-654 mice were administrated with pacDNA bearing a full locked nucleic acid (LNA) SSO targeting the aberrant splice site (pacDNA-654, 1.0 μmol kg 1, SSO basis), vehicle (phosphate buffered saline, PBS), or arginine-rich cell-penetrating peptide conjugated SSO (B pep-654, 1.0 μmol kg 1, SSO basis) (see Ref. 24) once daily for consecutive four days (FIG. 12). Animals were euthanized one week after the last administration, and the RNA was extracted from skin tissues for reverse transcriptase-PCR (RT-PCR) analysis. pacDNA-treated mice exhibited partially restored splicing in the skin (about 20%), indicating that skin-accumulated pacDNA can enter cells and engage with intracellular targets, while both controls showed no detectable EGFP mRNA (FIG. 4D). This result also demonstrates that the pacDNA can be designed to address targets within the cell nucleus, despite the fact the pacDNA is a non-cleavable conjugate with a molecular weight of about 300 kDa.

### Example 4

### Psoriasis Mouse Model

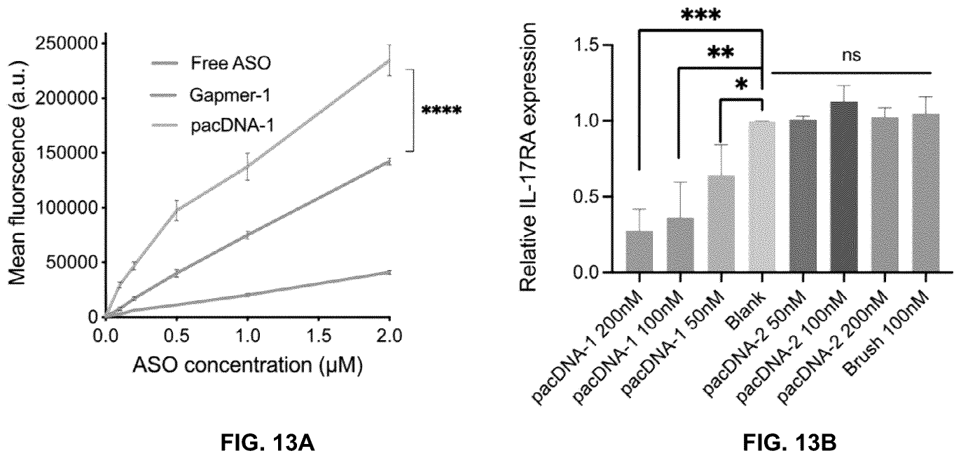

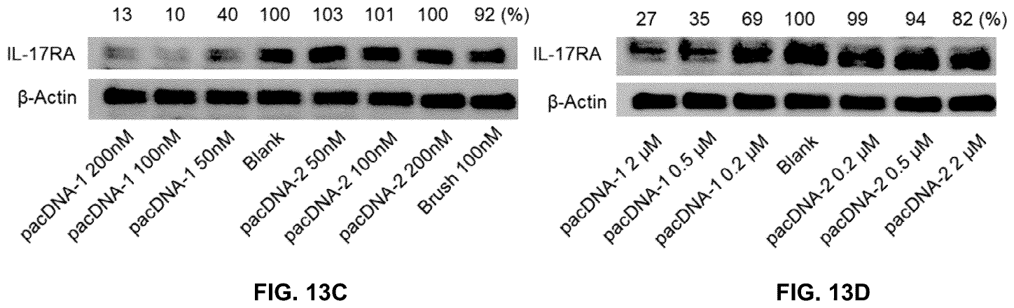

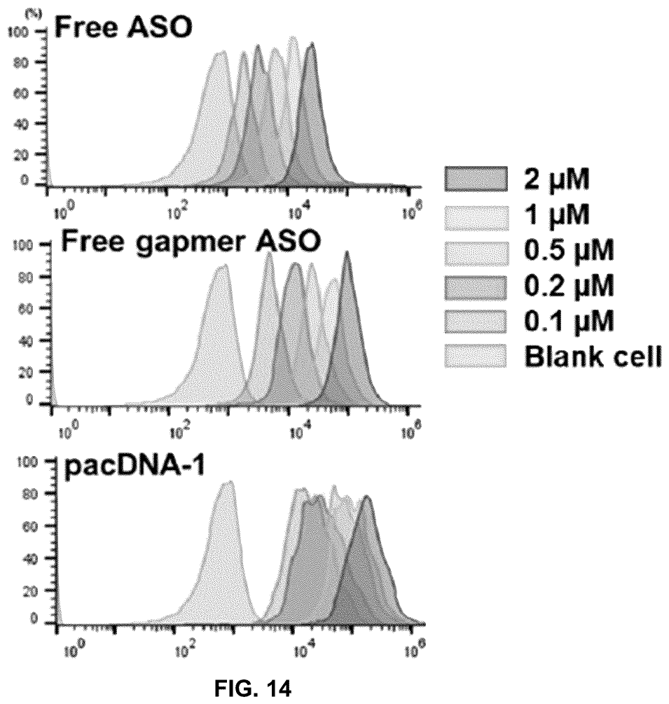

Next, the activity of pacDNA was explored using psoriasis as a model disease. pacDNA targeting IL-17RA (pacDNA-1) was synthesized and its cellular uptake investigated using NIH/3T3 mouse fibroblasts. Cells were treated with Cy5-labeled pacDNA-1 or two naked ASOs: a gapmer DNA with flanking PS and 2′ OMe modifications (Tables 3-4) and an unmodified DNA ASO. Flow cytometry showed that pacDNA-1 bearing the gapmer sequence exhibited higher cell uptake, approximately ten-fold greater than unmodified ASO and approximately four-fold higher than the gapmer at 0.1 μm (FIG. 13A; FIG. 14). To measure antisense activity, IL-17RA protein expression levels from NIH/3T3 and RAW 264.7 cells were measured by western blot after cells were treated with pacDNA-1, scrambled pacDNA control (pacDNA-2), and free bottlebrush polymer (FIGS. 13 B, 13C, 13D). It was observed that pacDNA-1 reduced IL-17RA expression in a dosage-dependent manner, with a maximum reduction level of about 70-90%, while negative control samples exhibited no detectable target depletion.

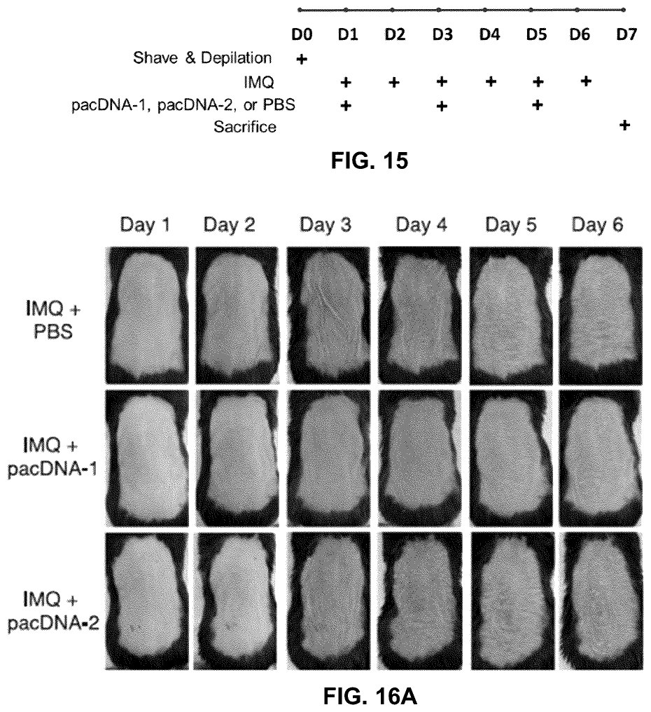

To test the in vivo efficacy of pacDNA-1 to suppress the development of the psoriasis-like phenotype after i.v. administration, an imiquimod (IMQ)-induced mouse model of psoriasis based upon C57BL/6 mice was used. Topical application of IMQ to the skin induces psoriasis-like dermatitis in mice with many characteristics of human psoriasis, including the formation of microabscesses, acanthosis, parakeratosis, hyperkeratosis, hyperplasia, erythema, and scaling (Ref. 25). IMQ cream (5%) was topically applied to shaved mouse dorsal skin daily for six consecutive days to induce psoriasis-like skin lesions. pacDNA-1, pacDNA-2, or vehicle (PBS) was injected through mouse tail vein (20 nmol ASO per animal) on days 1, 3, and 5 (FIG. 15). To examine the severity of the phenotype, the Psoriasis Area and Severity Index (PASI) was used, which is formulated by evaluating the degree of erythema (redness), induration (thickness), and desquamation (scaling) of the affected skin area.

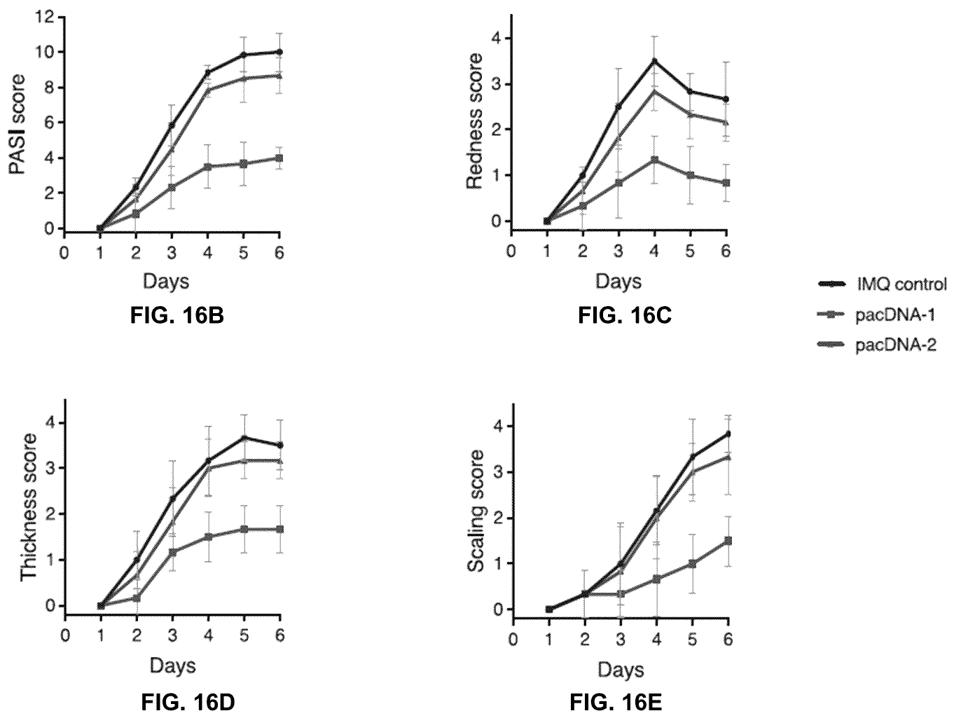

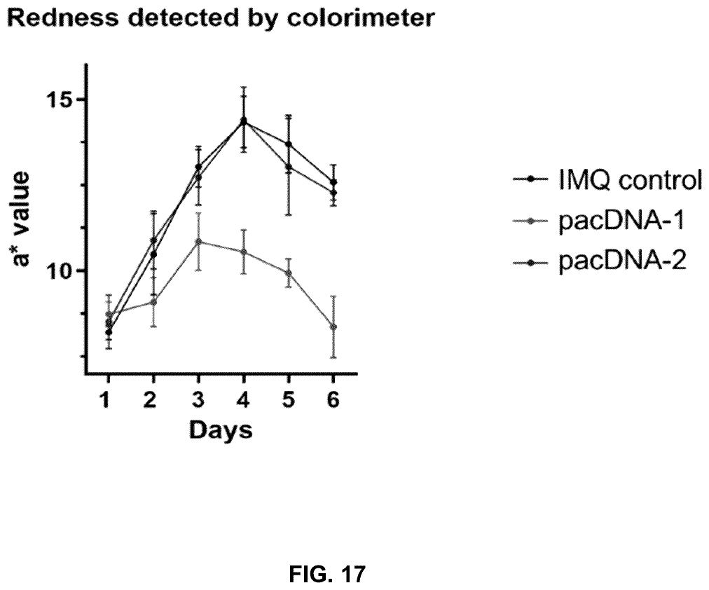

The development of psoriasis-like dermatitis from pacDNA-1-treated group was evidently suppressed when compared to pacDNA-2 and vehicle groups (FIG. 16A). Mice in the pacDNA-1-treated group showed a marked decrease in overall PASI score (60%, FIG. 16B) with redness (69%, FIG. 16C), thickness (52%, FIG. 16D), and scaling (61%, FIG. 16E) separately compared to vehicle-treated group, indicating the efficacy of pacDNA-1 in alleviating the psoriasis-like changes. In addition to the PASI scoring, the degree of skin erythema (i.e., redness of hemoglobin) (see Ref. 26) was also quantitatively evaluated by a tristimulus colorimeter and shown as “a*” of CIEL*a*b* (CIELAB) color space, which has been frequently used as an indicator of skin erythema (Ref. 27). The a* values of mouse dorsal skin, which were recorded daily (FIG. 17), also show a pronounced reduction in redness for the pacDNA-1-treated mice compared to negative control groups. Remarkably, pacDNA-1 was dosed at 5 mg kg 1 (ASO mass only), which is significantly lower than what is typical of this drug modality (on the order of tens to >100 mg kg 1). The notable bioactivity at such low dosages may be attributable to the evasion of renal clearance and greatly increased accumulation in the site of pathology.

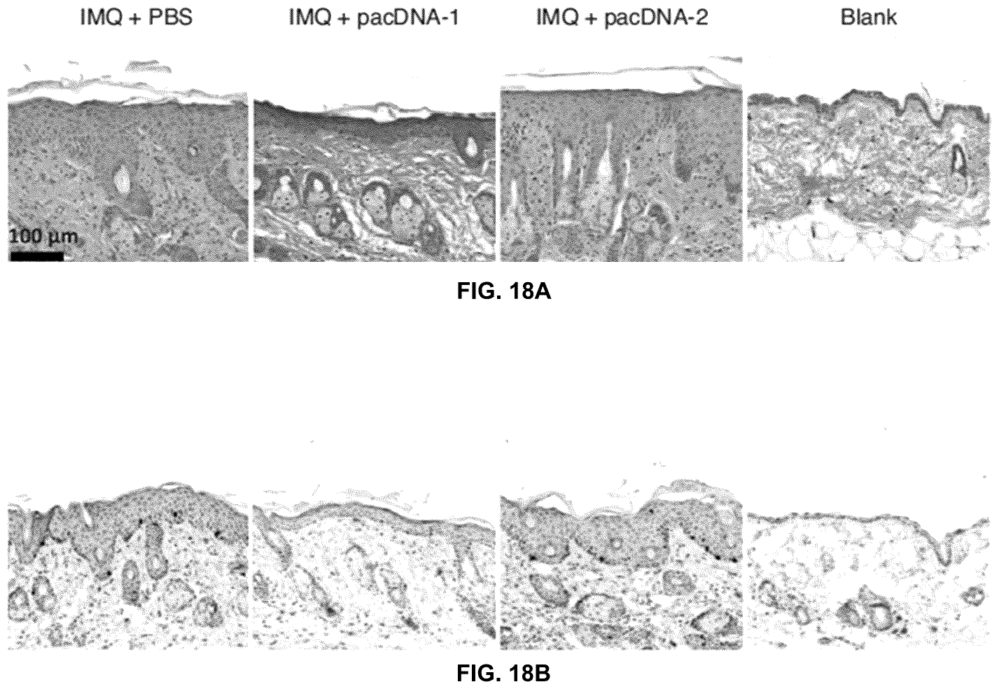

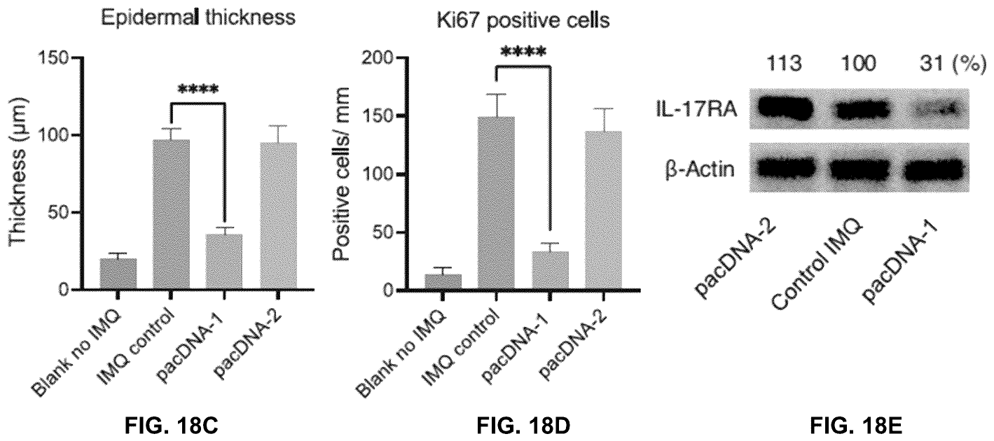

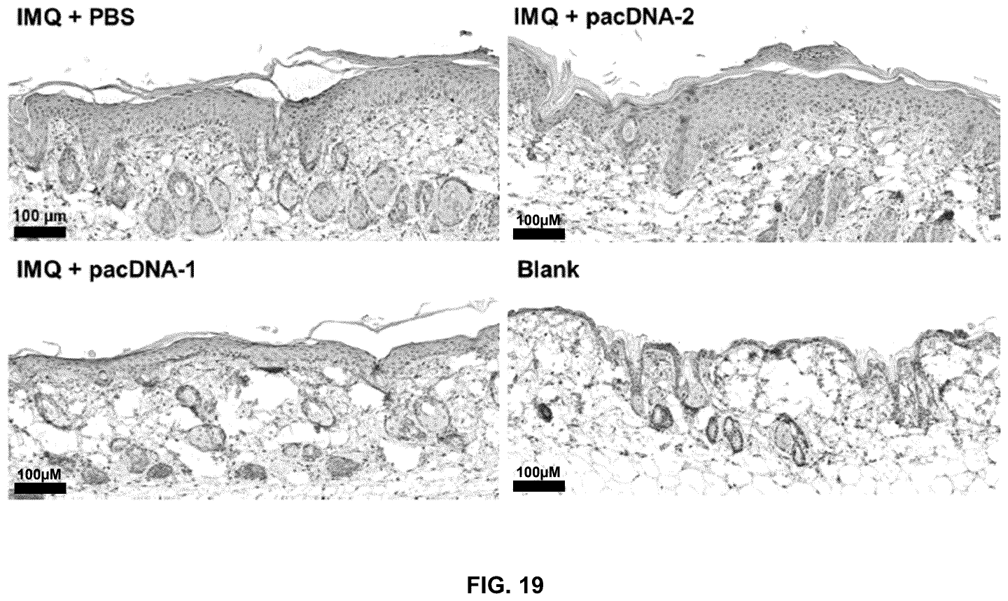

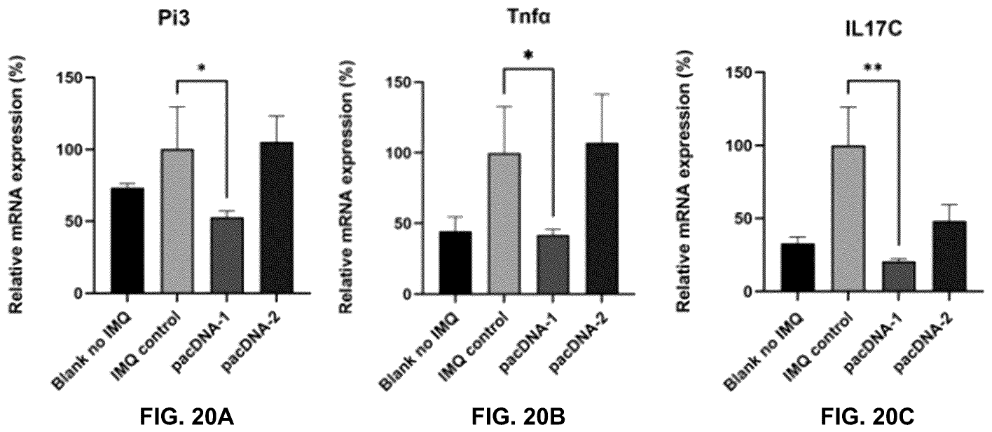

To further investigate the effect of pacDNA-1 on the tissue, cell, and biochemical levels, mouse dorsal skin was harvested on day 7 and evaluated for histological features and IL-17RA expression. Comparing with the vehicle and pacDNA-2-treated groups, the extent of these psoriatic histological characteristics of skin from the pacDNA-1-treated group were marked reduced (FIG. 18A). In particular, epidermal thickness decreased by 53% compared to the PBS control group (FIG. 18C). In addition, the number of Ki-67-positive cells at basal layers was decreased by 78% with the treatment of pacDNA-1 (FIGS. 18B, 18D). Overexpression of Ki-67 protein is associated with abnormal keratinocyte proliferation, a characteristic of psoriasis (Ref. 28). Finally, the expression of IL-17RA was directly determined by western blot using homogenized skin cells and immunohistochemical staining (FIG. 19). Band densitometry analysis of western blot bands shows that skin tissue from pacDNA-1-treated group express 69% less IL-17RA protein compared to the PBS control (FIG. 18E). Down-regulation of IL-17RA is associated with significantly decreased the transcript level of several psoriasis-related markers including Serpina1c (Pi3) (46% reduction, FIG. 20A), Tnfα (61% reduction, FIG. 20B), and IL-17C (57% reduction, FIG. 20C) compared to the pacDNA-2 treatment group.

### Example 5

### Materials and Experimental Procedures

### Materials

Phosphoramidites and supplies for oligonucleotide synthesis were purchased from Glen Research Co. (Sterling, VA, USA). RAW 264.7 macrophage-like mouse and NIH/3T3 mouse embryonic fibroblast cell lines were purchased from American Type Culture Collection (Rockville, MD, USA). @-Amine PEG methyl ether (number average molar mass (Mn)=10 kDa, polydispersity index (PDI)=1.05) was purchased from JenKem Technology (Plano, TX, USA). All other common materials were obtained from Sigma-Aldrich Inc. (St. Louis, MO, USA), Fisher Scientific Inc. (Waltham, MA, USA), or VWR International LLC (Radnor, PA, USA), and were used as received unless otherwise indicated. For the purification of oligonucleotide, reverse-phase high-performance liquid chromatography (HPLC) was performed on a Waters (Waters Co., Milford, MA, USA) Breeze 2 HPLC system coupled to a Symmetry® C18 3.5 μm, 4.6×75 mm reversed-phase column and a 2998 photodiode array (PDA) detector, using tricthylammonium acetate (TEAA) buffer (0.1 M) and HPLC-grade acetonitrile as mobile phases. N,N-Dimethylformamide (DMF) gel permeation chromatography (GPC) was carried out on a TOSOH EcoSEC HLC-8320 GPC system (Tokyo, Japan) equipped with a TSKgel® Alpha-M, 7.8 mm ID x 30 cm column and refractive index/ultraviolet-visible (RI/UV-Vis) detectors. HPLC-grade DMF with 0.05 M LiBr was used as the mobile phase, and samples were run at a flow rate of 0.4 mL/min. Aqueous GPC measurements were carried out on a Waters Breeze 2 GPC system equipped with an Ultrahydrogel™ 1000, 7.8×30 cm column and three Ultrahydrogel™ 250, 7.8×30 cm columns and a 2998 PDA detector for the separation of as-synthesized polymers from monomers/oligonucleotides. Matrix assisted laser desorption ionization-time of flight mass spectrometry (MALDI-TOF MS) measurements were performed on a Bruker Microflex LT mass spectrometer (Bruker Daltonics Inc., Billerica, MA, USA).

### Oligonucleotide Synthesis

Oligonucleotides including modifications were synthesized on a Model 391 DNA synthesizer (Applied Biosystems, Inc., Foster City, CA, USA) using standard solid-phase phosphoramidite methodology. DNA strands were cleaved from the controlled pore glass (CPG) solid support using aqueous ammonium hydroxide (28-30% NH3 basis) at room temperature for 18 hours. Gapmers and locked nucleic acid (LNA)-modified strands were synthesized on Universal Support III PS CPG, cleaved by treating with 2 M ammonia in methanol at RT for 60 min, and deprotected using aqueous ammonium hydroxide (28-30% NH3 basis) at room temperature for 18 h. All strands were purified by reverse-phase HPLC liquid chromatography. The successful synthesis of all sequences was verified by MALDI-TOF MS.

### Synthesis of Diblock Bottlebrush Polymer.

Two monomers, norbornenyl bromide (compound 1) and norbornenyl PEG (compound 2) were synthesized following previously published method (Ref. 29). Norbornenyl bromide (15 μmol, 5 equiv.) was dissolved in anhydrous toluene and placed in a Schlenk flask under N₂, which was then vacuumed and purged with N₂ gas three times. Then, a solution of Grubbs' catalyst 3rd generation (3 μmol, 1 equiv.) in deoxygenated toluene was rapidly added by gastight syringe. The reaction mixture was stirred for 30 min at room temperature followed by addition of a mixed solution (5 mL of toluene and 4.5 mL of dichloromethane) containing norbornenyl PEG (105 μmol, 35 equiv.) with a gastight syringe. The reaction mixture was further stirred for 2 hours at room temperature. Thereafter, several drops of ethyl vinyl ether (EVE) were added into the Schlenk flask, and the reaction mixture was further stirred for 30 minutes. The mixture was then concentrated and precipitated (3×) into cold diethyl ether to give a white solid polymer (compound 3). The polymer was dried under high vacuum, redissolved in DMF, and mixed with an excess of sodium azide while stirring. After overnight reaction at room temperature, the materials were transferred to a dialysis tubing (molecular weight cut-off (MWCO), 6-8 kDa), dialyzed against NANOPURE® bacterial- and particle-free ultrapure water for 24 hours, and the dried by lyophilization to give azide-functionalized polymer (compound 4). DMF-GPC analysis determined the bottlebrush polymer with number average molar mass (Mn) of about 290 kDa, molecular weight (Mw) of about 360 kDa, polydispersity index (PDI) of about 1.20, and a yield of ˜93%. To quantify the number of azide groups per copolymer available for coupling, 10 nmol of the polymer was dissolved in 400 μL of DMF and conjugated with dibenzocyclooctyne (DBCO)-modified Cyanine 5 (Lumiprobe Co., Hunt Valley, MD, USA; 1000 nmol). The reaction mixture was gently shaken on an Eppendorf Thermomixer at room temperature overnight. Thereafter, the solution was dialyzed against a NANOPURE® water using dialysis tubing with a MWCO of 6-8 kDa for 72 hours. The UV-Vis absorption of the polymer solution at 646 nm was measured and compared with a standard curve. The number of Cy5 molecules per polymer was calculated based on the known polymer concentration. Approximately 4.9 Cy5 tags were attached to each diblock (pNBr₅-bpNPEG₂₉) brush copolymer.

### Synthesis of Cy5-Labeled Bottlebrush Polymer

The bottlebrush polymer was labelled with Cy5 via copper-catalyzed click chemistry. The brush polymer (15 mg, 50 nmol) in NANOPURE® water (2 mL) was added with Cy5-alkyne (100 nmol, 100 μL 1 mM DMSO solution). The catalyst system (CuSO4·5H2O, 40 nmol; Tris-hydroxypropyltriazolylmethylamine, 50 nmol; sodium ascorbate, 250 nmol) was added to the solution and stirred at room temperature overnight. The reaction mixture was dialyzed against NANOPURE® water and further purified using aqueous GPC. The fractions containing the conjugate were collected, concentrated, desalted, and lyophilized to afford a blue powder.

Synthesis of pacDNAs

In a typical procedure, azide-functionalized bottlebrush polymer (compound 4) (15 mg, 50 nmol) was dissolved in 800 μL of aqueous NaCl solution (2 M), to which DBCO-modified gapmer (100 nmol) or DBCO-modified SSO-654 was added (2 equiv. to N3, dissolved in 200 μL of NaCl aqueous solution). The reaction mixtures were shaken gently for 17 hours at 50° C. on an Eppendorf Thermomixer. Thereafter, the conjugation product was isolated using aqueous GPC. The conjugates were desalted using a NAP-25 gravity flow purification column and subsequently lyophilized to yield a white powder (or blue powders for Cy5-labeled pacDNA). The number of ASO strands per brush were detected using ASO UV absorbance at 280/260 nm. Specifically, 4.2 mg of the pacDNA was weighted and dissolved in 200 μL of NANOPURE® water (65 nmol/mL). The concentration of ASO was measured to be 125.13 nmol/mL using a NANODROP® spectrophotometer device, indicating about 1.92 ASO per brush.

Synthesis of B pep-654.

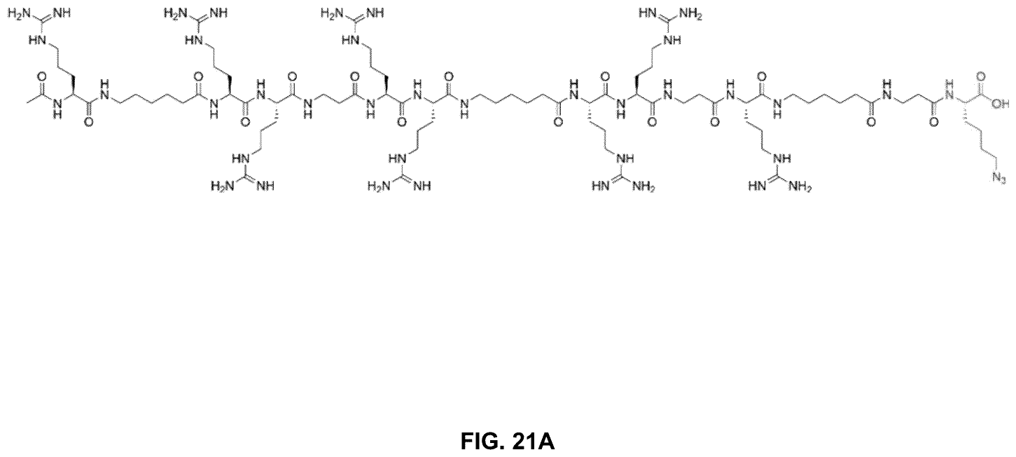

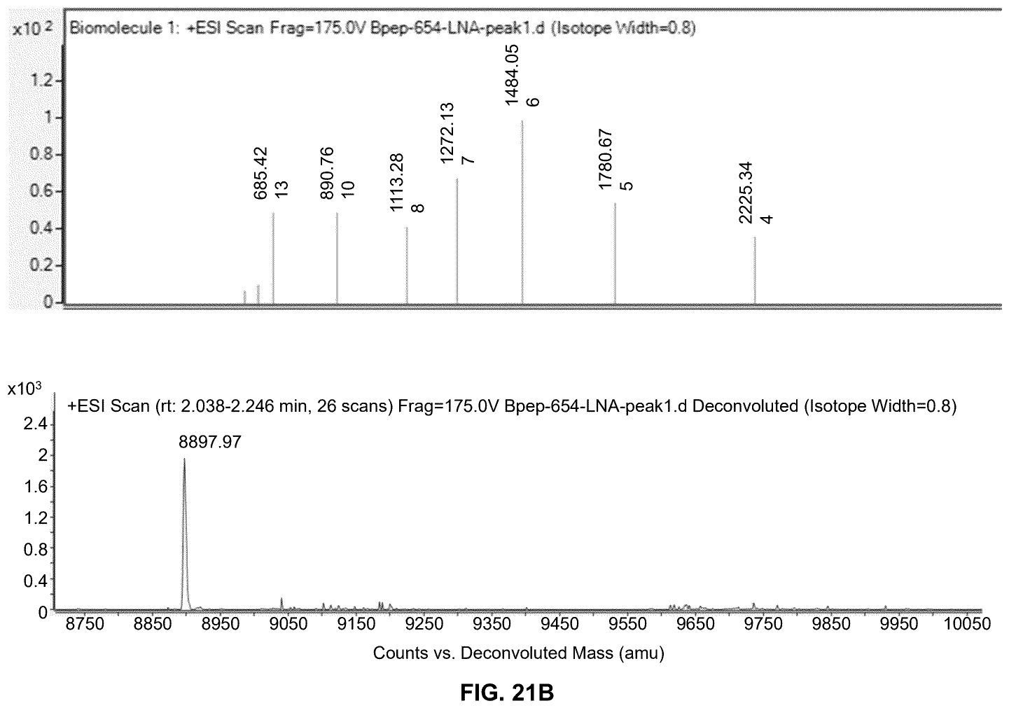

DBCO-modified (100 nmol)SSO-654 and C-terminus azide-functionalized B-peptide (200 nmol, SEQ ID NO: 17: Ac-RXRRBRRXRRBRXBK (N3), 6-aminohexanoic acid (X), β-alanine (B)) were dissolved in 1500 μL of aqueous NaCl solution (2 M). The reaction mixture was shaken gently for 17 hours at 50° C. on an Eppendorf Thermomixer. Thereafter, the conjugation product was purified by reverse-phase HPLC liquid chromatography, and the successful synthesis of conjugate was confirmed by liquid chromatography-electrospray ionization mass spectrometry (LC-ESI-MS) (Calculated: 8897 Da; Measured 8897.97 Da, FIGS. 21A, 21B). Purified B pep-654 conjugate was desalted with a NAP-25 column, stored as lyophilized powder, and kept at −20° C.

### Cell Culture

RAW 264.7 macrophage cells and NIH/3T3 fibroblast cells were cultured in full-growth medium comprising Dulbecco's Modified Eagle's Medium and supplied with 10% fetal bovine serum (FBS), 100 units/mL penicillin, and 100 μg/mL streptomycin at 37° C. in a humidified atmosphere containing 5% CO2. The culture medium was changed every two days. The cells were harvested with 0.25% trypsin and 2.21 mM ethylenediamine tetraacetic acid (EDTA) through trypsinization.

### Cellular Uptake Determined by Flow Cytometry

NIH/3T3 cells were seeded into 24-well plates at 5.0×10⁵ cells per well in 1 mL of full-growth medium and cultured overnight at 37° C. with 5% CO₂. After removing the medium, the cells were rinsed with PBS 3× and then serum-free medium containing Cy5-labeled pacDNA-1, free gapmer-1, or free unmodified ASO with varying doses (0.1 to 2 μM, ASO basis) were added to each well. The cells were further incubated at 37° C. for 4 hours. Subsequently, medium was removed, the cells were washed with PBS 3×, trypsinized, centrifuged, and re-suspended in 600 μL of fresh PBS. All samples were analyzed by flow cytometry (Attune N×T flow cytometer, Invitrogen, Carlsbad, CA, USA) to determine the extent of cell uptake.

### Western Blot Analysis of Raw-264.7 and NIH 3T3 Cell Lines

The gene regulation efficacy of pacDNA-1 in vitro was evaluated in two cell lines using western blot.

Raw-264.7 macrophage cells were seeded into 24-well plates at 5.0×10⁴ cells per well in 1 mL of full-growth medium and cultured overnight at 37° C. with 5% CO₂. Cells were then incubated with pacDNA-1 (50 to 200 nM, ASO basis)), pacDNA-2, or brush polymer in 500 μL of serum-free medium for 6 hours.

NIH/3T3 cells were seeded into 24-well plates at 1.0×10⁵ cells per well in 1 mL of full-growth medium and cultured overnight at 37° C. with 5% CO₂. Cells were then incubated with pacDNA-1 (0.2 to 2 μM, ASO basis), or pacDNA-2 in 500 μL of serum-free medium for 6 hours. Thereafter, 500 μL of full-growth medium was added into the wells and cells were further cultured for another 66 hours. Whole cell lysate was collected in 60 μL of radioimmunoprecipitation assay cell lysis buffer (RIPA) containing Halt™ protease and phosphatase inhibitor cocktail, and 5 mM of EDTA (Thermo Fisher Scientific, Waltham, MA, USA). Protein concentrations were measured by a bicinchoninic acid (BCA) protein assay with bovine serum albumin (BSA) as the protein standard. Equal amounts (20 ug/lane) of protein samples were separated on 4-20% gradient sodium dodecyl sulfate-polyacrylamide gel electrophoresis (SDS-PAGE), and electro-transferred to nitrocellulose membrane. The membranes were blocked for 1 hour at room temperature with 3% bovine serum albumin (BSA) in tris-buffered saline supplemented with 0.05% Tween 20, then incubated with primary antibodies against B-actin (1:2000 dilution, Thermo Fisher Scientific, Waltham, MA, USA) and IL-17RA (1:500 dilution, Invitrogen, Carlsbad, CA, USA) at 4° C. overnight. After washing and incubation with anti-rabbit secondary antibody (1:2500 dilution, Cell Signaling Technology, Danvers, MA, USA) and anti-mouse secondary antibody (1:5000 dilution, Cell Signaling Technology, Danvers, MA, USA) at room temperature for 1 hour, protein bands were visualized by using an enhanced chemiluminescence (ECL) Western Blotting Substrate (Pierce, Thermo Fisher Scientific, Waltham, MA, USA).

### Animal Studies

All animal protocols were approved by the Institutional Animal Care and Use Committee of Northeastern University (Boston, MA, USA) and carried out under pathogen-free conditions in the animal facility of Northeastern University and in accordance with National Institutes of Health (NIH) animal care guidelines. Female C57BL/6 mice (6-8 weeks old) were purchased from Charles River Laboratories. Tg(CAG-EGFP*) 1Rkol/RjulJ (EGFP-654) mice (cryorecovered) were purchased from The Jackson Laboratory. The animals were given free access to a standard laboratory diet and water and were kept in the laboratory animal facility with temperature and relative humidity maintained at 23±2° C. and 50±20%, respectively, under a 12 hour: 12 hour light: dark cycle. Mice were given at least one week to acclimatize new environment and housing conditions of the animal facility prior to experiments.

### Ex Vivo Organ Imaging and Fluorescence Intensity Measurement in Different Tissues

Cy5-labeled brush polymer at a dose of 0.5 μmol/kg animal weight was intravenously injected into the C57BL/6 mice. After 24 hours, mice were sacrificed, and skin and other major organs were harvested for fluorescence imaging using an IVIS Lumina II imaging system (Caliper Life Sciences, Inc. MA, USA). To measure the fluorescence intensity in tissue homogenate, the experimental procedure was based upon a previously described method with modifications. Tissues from treated and naïve mice were homogenized in homogenization buffer (10 mM Tris pH 7.4 and 0.5% Triton X-100) at a ratio of 100 mg of tissue per mL. 50 μL of tissue homogenates from each organ of brush polymer-treated mice were then transferred to a 96-well plate and imaged with the IVIS imaging system. Tissue homogenate from naïve mice was mixed with Cy5-labeled brush polymer at different concentrations (0.05 to 0.2 nmol/mL) to create standard curve. The total radiance of the selected region of interest was measured using Living Image software.

### Ex Vivo Organ Imaging

PBS buffer, Cy5-labeled pacDNA-1, or Cy5-labeled free gapmer-1 at a dose of 1.0 μmol/kg animal weight (ASO basis) was intravenously injected into the C57BL/6 mice. After 24 hours, mice were sacrificed, the animal skins from three groups were harvested for fluorescence imaging using an IVIS Lumina II imaging system (Caliper Life Sciences, Inc. MA, USA).

### Fluorescence Microscopy of Sectioned Mouse Skin

C57BL/6 mice were i.v. injected with Cy5-labeled brush polymer at a dose of 0.5 μmol/kg animal weight or Cy5-labeled pacDNA-1 (1.0 μmol/kg, ASO basis). After 24 hours, skin tissues were immediately frozen in optimal cutting temperature (O.C.T.) compound (Fisher Scientific Inc., Waltham, MA, USA), sectioned, stained with Hoechst 33342 DNA-specific fluorescent stain (2′-[4-ethoxyphenyl]-5-[4-methyl-1-piperazinyl]-2,5′-bi-1H-benzimidazole trihydrochloride trihydrate), and imaged on an LSM-800 confocal laser scanning microscope (Carl Zeiss Ltd., Cambridge, UK).

### Immunofluorescence Staining.

For immunofluorescence analysis, cryostat-cut sections of dorsal skin from brush polymer-treated mice were fixed in 4% paraformaldehyde for 20 minutes, then blocked with 2.5% normal goat serum for 30 minutes. Sections were then stained with primary antibody against F4/80 (targeting macrophages, 1:200 dilution, Cell Signaling Technology, 70076), CD11c (targeting dermal dendritic cells, 1:100 dilution, Cell Signaling Technology, Cat. No. 97585), vimentin (targeting fibroblasts, 1:200 dilution, Cell Signaling Technology, Cat. No. 5741), or perilipin-1 (targeting adipose cells, 1:50 dilution, Cell Signaling Technology, Cat. No. 9349) followed by incubated with Alexa Fluor 555-labeled goat anti-rabbit IgG secondary antibody (1:500 dilution, Thermo Fisher Scientific, Waltham, MA, USA, Cat. No. A-21429) for 1 hour. Samples were stained with DAPI (common name, 4′,6-diamidino-2-phenylindole; IUPAC name, 2-(4-carbamimidoylphenyl)-1H-indole-6-carboximidamide) for 10 minutes and imaged on an LSM-800 confocal laser scanning microscope (Carl Zeiss Ltd., Cambridge, UK). Imaging settings were kept identical for all samples.

### Egfp-654 Animal Treatment

6-week-old female EGFP-654 mice were divided into three groups: pacDNA-654 (i.v. injection), B pep-654 (i.v. injection), and vehicle (PBS, i.v. injection). Animals were treated once a day for consecutive four days for a total of four doses at dosage of 1.0 μmol/kg for pacDNA-654, 1.0 μmol/kg for B pep-654, or vehicle only. Animals were euthanized by CO2 inhalation at one week post last administration, and skin tissues were collected and immediately frozen for RNA isolation.

### RNA Isolation and Analysis of EGFP-654 Mouse Skin Splicing Correction

Frozen mouse skin tissue from different groups (around 30 mg) was subjected to homogenization using the BeadBlaster 24R Refrigerated Homogenizer (Benchmark Scientific, Sayreville, NJ, USA). The homogenization process involved operating the homogenizer at a speed of 3650 revolutions per minute (rpm) for 30 seconds, with a linear speed of 6.00 m/s, and maintaining a temperature of 4° C. Two homogenization cycles were performed, with a 30 second interval between each cycle. Total RNA was extracted from the homogenized tissue using the RNEASY® Fibrous Tissue Mini Kit (Qiagen, Germantown, MD, USA) following standard procedures recommended by the manufacturer. The EGFP mRNA was amplified by one-step RT-PCR using SuperScript™ IV One-Step RT-PCR System (Invitrogen) following the recommended protocol. In this method, 100 ng of isolated RNA was used for RT-PCR reaction, which proceeded as follows: 55° C. for 10 minutes, 98° C. for 2 minutes, followed by 40 cycles of amplification at 98° C. for 10 seconds, 68° C. for 10 seconds, 72° C. for 15 seconds, and a final extension was performed at 72° C. for 5 minutes. The resulting PCR products were separated on a 4-20% gradient nondenaturing polyacrylamide gel. To visualize the bands, the gel was stained with GelRed Nucleic Acid Gel Stain in 0.1M NaCl solution for 30 minutes (Biotium, Inc., Fremont, CA, USA). EGFP forward primer: 5′-CGTAAACGGCCACAAGTTCAGCG-3′ (SEQ ID NO: 18), reverse primer: 5′-GTGGTGCAGATGAACTTCAGGGTC-3′ (SEQ ID NO: 19).

### Induction of Psoriasis-Like Skin by Imiquimod (IMQ)

The dorsal hair of C57BL/6 mice was removed, exposing a skin surface area of about 2 cm×3 cm based on previously published protocols with modifications (Ref. 30). After 24 hours, 5% imiquimod (IMQ) cream (Taro Pharmaceutical Industries, Ltd., Hawthorne, NY, USA) was applied to the exposed back skin daily at a dose of 62.5 mg for six consecutive days to develop a psoriasis-like dermatitis.

### Treatment in IMQ-Induced Psoriasis-Like Mouse Skin

Mice were divided into three groups: IMQ+pacDNA-1, IMQ+pacDNA-2, and IMQ+vehicle only (PBS buffer). The pacDNA-1 or pacDNA-2 was i.v. injected to the mice (20 nmol ASO per animal) on day 1, day 3, and day 5. Vehicle (PBS buffer, 200 μL) was intravenously administrated to mice as control. On day 7, all mice were sacrificed, and dorsal skin tissue were collected for further studies.

### Scoring Severity of Skin Inflammation

The severity of psoriasis-like skin condition was daily graded using modified Psoriasis Area and Severity Index (PASI) scoring system. Skin erythema, scale, and thickness were scored independently on a four-point scale according to the degree of the inflammation: 0, none; 1, slight: 2, moderate; 3, marked; 4, very marked. The modified PASI score was calculated as a cumulative score for these three parameters (scale 0-12).

### Measurement of Mouse Dorsal Skin Redness

The redness (a* value) of mouse dorsal skin was quantified through L*a*b colorimetry using a tristimulus colorimeter daily. The back region of each mouse was photographed under controlled lighting conditions and identical camera settings.

Quantification of mRNA Level by Quantitative Real-Time PCR (qRT-PCR)

Frozen mouse dorsal skin tissue from different groups was grinded using a tapered tissue grinder, and the total RNA was extracted with the RNEASY® Fibrous Tissue Mini Kit (Qiagen, Germantown, MD, USA) following manufacture-suggested protocols. The RNA concentration was determined using a NANODROP® 2000 spectrophotometer (Thermo Fisher Scientific, Waltham, MA, USA). Total RNA (300 ng) was reverse transcribed to cDNA using the iScript™ Advanced cDNA Synthesis Kit at 42° C. for 20 min and 95° C. for 1 minutes. The cDNA was amplified with Magic SYBR® Mixture (CoWin Biosciences (CWBIO), Boston, MA, USA). The results were normalized to glyceraldehyde-3-phosphate dehydrogenase (Gapdh) expression. Each data point is the mean of three replicate experiments, each performed in triplicate. The pre-designed primers were purchased from Integrated DNA Technology (Coralville, IA, USA); PrimeTime™ Predesigned qPCR Assay ID Nos: Mouse: Tnfα, Mm.PT.58.12575861; Pi3, Mm.PT.58.43208182; IL-17C, Mm.PT.58.30030128; Gapdh, Mm.PT.39a.1. Determinations of relative gene expression changes were carried out according to the 2-AsCt method.

### Histology Analysis

Dorsal skin tissue samples from treated mice and naïve mice were collected, placed in 10% formalin neutral buffer solution, and embedded in paraffin. Histopathology (hematoxylin and eosin (H&E) staining) and immunohistochemistry studies (IL-17RA and Ki67) were carried out at iHisto Inc. according to standard protocol.

Western Blot Analysis with IMQ-Induced Psoriasis-Like Skin Tissue

Fresh frozen dorsal skin tissues from treated mice were lysed with lysis buffer and grinded using a tapered tissue grinder at 4° C. to prepared protein extracts. After centrifugation at 14,000 rpm at 4° C. for 20 min, supernatants were collected and protein concentration was determined. Western blot analysis was carried out according to the same procedure described for IL-17RA protein expression in Raw-264.7 and NIH/3T3 cell lines described above.

### REFERENCES

References cited throughout the application are listed below. In some instances, a single reference number (Ref.) refers to more than one reference.
1)
a) E. Sutterby, P. Thurgood, S. Baratchi, K. Khoshmanesh, E. Pirogova, Small 2020, 16, 2002515
b) E. A. Sykes, Q. Dai, K. M. Tsoi, D. M. Hwang, W. C. Chan, Nat. Commun. 2014, 5, 3796.
2)
a) H. Wang, W. Sheng, Nanoscale Res. Lett 2017, 12, 365
b) C. Alric, Miladi, D. Kryza, J. Taleb, F. Lux, R. Bazzi, C. Billotey, M. Janier, P. Perriat, S. Roux, O. Tillement, Nanoscale 2013, 5, 5930.
3)
a) F. Jia, X. Lu, D. Wang, X. Cao, X. Tan, H. Lu, K. Zhang, J. Am. Chem. Soc. 2017, 139, 10605
b) X. Lu, T.-H. Tran, F. Jia, X. Tan, S. Davis, S. Krishnan, M. M. Amiji, K. Zhang, J. Am. Chem. Soc. 2015, 137, 12466
c) Y. Wang, D. Wang, F. Jia, A. Miller, X. Tan, P. Chen, L. Zhang, H. Lu, Y. Fang, X. Kang, J. Cai, M. Ren, K. Zhang, ACS Appl. Mater. Interfaces 2020, 12, 45830
d) F. Jia, P. Chen, D. Wang, Y. Sun, M. Ren, Y. Wang, X. Cao, L. Zhang, Y. Fang, X. Tan, H. Lu, J. Cai, X. Lu, K. Zhang, ACS Appl. Mater. Interfaces 2021, 13, 42533
e) Y. Fang, K. Zhang, Encyclopedia of Nanomaterials, 1st Ed., (Eds: Y. Yin, Y. Lu, Y. Xia), Elsevier, 2023, pp. 574-589.
f) L. Zhang, Y. Wang, P. Chen, D. Wang, T. Sun, Z. Zhang, R. Wang, X. Kang, Y. Fang, H. Lu, J. Cai, M. Ren, S. S. Dong, K. Zhang, RSC Chem Biol 2023, 4, 138.
4) Y. Wang, D. Wang, J. Lin, Z. Lyu, P. Chen, T. Sun, C. Xue, M. Mojtabavi, A. Vedadghavami, Z. Zhang, R. Wang, L. Zhang, C. Park, G. S. Heo, Y. Liu, S. S. Dong, K. Zhang, Angew. Chem., Int. Ed. 2022, 61, 202204576.
5) D. Wang, J. Lin, F. Jia, X. Tan, Y. Wang, X. Sun, X. Cao, F. Che, H. Lu, X. Gao, J. C. Shimkonis, Z. Nyoni, X. Lu, K. Zhang, Sci. Adv. 5, eaav9322.
6)
a) S. K. Ippagunta, R. Gangwar, D. Finkelstein, P. Vogel, S. Pelletier, S. Gingras, V. Redecke, H. Häcker, Proc. Natl. Acad. Sci. USA 2016, 113, E6162
b) K. T. Lewandowski, R. Thiede, N. Guido, W. L. Daniel, R. Kang, M. I. Guerrero-Zayas, M. A. Seeger, X. Q. Wang, D. A. Giljohann, A. S. Paller, J. Invest. Dermatol. 2017, 137, 2027.
7) B. Li, L. Huang, P. Lv, X. Li, G. Liu, Y. Chen, Z. Wang, X. Qian, Y. Shen, Y. Li, W. Fang, Immunol. Res. 2020, 68, 296.
8) A. Blauvelt, J. Invest. Dermatol 2008, 128, 1064.
9) E. Toussirot, Expert Opin. Biol. Ther. 2018, 18, 101.
10) K. Reich, A. Blauvelt, A. Armstrong, R. G. Langley, A. de Vera, F. Kolbinger, S. Spindeldreher, M. Ren, G. Bruin, J. Eur. Acad. Dermatol. Venereol. 2019, 33, 1733.
11) L. Puig, Drugs Today (Barc) 2017, 53, 283.
12) C. Jeon, S. Sekhon, D. Yan, L. Afifi, M. Nakamura, T. Bhutani, Hum. Vaccines Immunother. 2017, 13, 2247.
13)
a) V. K. Rapalli, T. Waghule, S. Gorantla, S. K. Dubey, R. N. Saha, G. Singhvi, Drug Discov. 2020, 25, 2212
b) H. Liu, R. S. Kang, K. Bagnowski, J. M. Yu, S. Radecki, W. L. Daniel, B. R. Anderson, S. Nallagatla, A. Schook, R. Agarwal, D. A. Giljohann, A. S. Paller, J. Invest. Dermatol. 2020, 140, 435
c) P. Sharma, A. Kumar, T. Agarwal, A. D. Dey, F. D. Moghaddam, I. Rahimmanesh, M. Ghovvati, S. Yousefiasl, A. Borzacchiello, A. Mohammadi, V. R. Yella, O. Moradi, E. Sharifi, Int. J. Biol. Macromol. 2022, 220, 920
d) M. Zakrewsky, S. Kumar, S. Mitragotri, J. Control Release 2015, 219, 445.
14)
a) B. Gurav, G. Srinivasan, Curr. Sci. 2017, 112, 490
b) Y. Weng, Q. Huang, C. Li, Y. Yang, X. Wang, J. Yu, Y. Huang, X.-J. Liang, Mol. Ther. Nucleic Acids 2020, 19, 581.
15) Y. Fang, J. Cai, M. Ren, T. Zhong, D. Wang, K. Zhang, ACS Nano 2024, 18, 592.
16)
a) Y. Fang, X. Lu, D. Wang, J. Cai, Y. Wang, P. Chen, M. Ren, H. Lu, J. Union, L. Zhang, Y. Sun, F. Jia, X. Kang, X. Tan, K. Zhang, J. Am. Chem. Soc. 2021, 143, 1296
b) P. Chen, D. Wang, Y. Wang, L. Zhang, Q. Wang, L. Liu, J. Li, X. Sun, M. Ren, R. Wang, Y. Fang, J. J. Zhao, K. Zhang, Nano Lett. 2022, 22, 4058.
17)
a) M. K. Bijsterbosch, E. T. Rump, R. L. A. D. Vrueh, R. Dorland, R. van Veghel, K. L. Tivel, E. A. L. Biessen, T. J. C. van Berkel, M. Manoharan, Nucleic Acids Res. 2000, 28,
b) R. S. Geary, R. Z. Yu, T. Watanabe, S. P. Henry, G. E. Hardee, A. Chappell, J. Matson, H. Sasmor, L. Cummins, A. A. Levin, Drug Metab. Dispos. 2003, 31, 1419.
18) D. C. Litzinger, J. M. Brown, I. Wala, S. A. Kaufman, G. Y. han, C. L. Farrell, D. Collins, Biochim. Biophys. Acta Biomembr. 1996, 1281, 139.
19) M. Dupasquier, P. Stoitzner, A. v. Oudenaren, N. Romani, P. J. M. Leenen, J. Invest. Dermatol. 2004, 123, 876.
20)
a) S.-S. Kim, S.-J. Gwak, C. Y. Choi, B.-S. Kim, J. Biomed. Mater. Res 2005, 75B, 369
b) F. Bernerd, D. Asselineau, Cell Death Differ. 1998, 5, 792
c) W. Mah, G. Jiang, D. Olver, C. Gallant-Behm, C. Wiebe, D. A. Hart, L. Koivisto, H. Larjava, L. Häkkinen, Am. J. Clin. Pathol. 2017, 187, 1717.
21) C. Miggitsch, A. Meryk, E. Naismith, L. Pangrazzi, A. Ejaz, B. Jenewein, S. Wagner, F. Nägele, G. Fenkart, K. Trieb, W. Zwerschke, B. Grubeck-Loebenstein, EBioMedicine 2019, 46,387.
22) P. Sazani, F. Gemignani, S.-H. Kang, M. A. Maier, M. Manoharan, M. Persmark, D. Bortner, R. Kole, Nat. Biotechnol 2002, 20, 1228.
23) J. Roberts, E. Palma, P. Sazani, H. Ørum, M. Cho, R. Kole, Mol. Ther. 2006, 14, 471.
24) N. B. Lu-Nguyen, S. A. Jarmin, A. F. Saleh, L. Popplewell, M. J. Gait, G. Dickson, Mol. Ther. 2015, 23, 1341.
25)
a) L. van der Fits, S. Mourits, J. S. Voerman, M. Kant, L. Boon, J. D. Laman, F. Cornelissen, A.-M. Mus, E. Florencia, E. P. Prens, E. Lubberts, J. Immunol. 2009, 182, 5836
b) C. E. Griffiths, J. N. Barker, Lancet 2007, 370, 263
c) M. W. Greaves, G. D. Weinstein, N. Engl. J. Med. 1995, 332, 581.
26) M. Baquié, B. Kasraee, Skin Res Technol 2014, 20, 218.
27)
a) J. C. Seitz, C. G. Whitmore, Dermatology (Basel) 1988, 177, 70
b) M. H. A. Fadzil, D. Ihtatho, A. Mohd Affandi, S. H. Hussein, J Med Eng Technol 2009, 33, 516.
28) L. Li, H.-y. Zhang, X.-q. Zhong, Y. Lu, J. Wei, L. Li, H. Chen, C. Lu, L. Han, Life Sci. 2020, 243, 117231.
29)
a) X. Lu, F. Jia, X. Tan, D. Wang, X. Cao, J. Zheng, K. Zhang, J. Am. Chem. Soc. 2016, 138, 9097-9100
b) J. K. Pontrello, M. J. Allen, E. S. Underbakke, L. L. Kiessling, J. Am. Chem. Soc. 2005, 127, 14536-14537.
30)
a) H. Shen, Y. Sha, J. Huang, A. Q. Mao, T. Zhang, M. Y. Wu, F. Sun, Y. Y. Yu, Z. Q. Cheng, Y. T. Gong, Am J Transl Res 2021, 13, 12626-12637
b) H. Guo, M. Li, H. Liu, in Int. J. Mol. Sci., Vol. 23, 2022.
31) T. Liu, S. Li, S. Ying, S. Tang, Y. Ding, Y. Li, J. Qiao, H. Fang, Front Immunol 2020, 11, 594735.
32) A. Rendon, K. Schäkel, Int J Mol Sci 2019, 20, 1475.
33) Y. Fang, J. Cai, F. Fei, T. Zhong, M. Ren, D. Wang, Y. Li, K. Zhang, Small 2024, 20, e2403949.

### INCORPORATION BY REFERENCE; EQUIVALENTS

The teachings of all manuscripts, patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method for treating or preventing a skin-related condition in a subject in need thereof, comprising administering to the subject a therapeutically effective amount of a composition comprising a bottlebrush polymer-oligonucleotide conjugate, wherein the conjugate comprises:
a polymer backbone,
polyethylene glycol (PEG) polymer arms covalently linked to the polymer backbone, and
an oligonucleotide covalently linked to the backbone, wherein the oligonucleotide modulates expression of one or more transcripts associated with the skin-related condition.

2. The method of claim 1, wherein the skin-related condition is selected from the group comprising: autoimmune skin diseases, skin cancers or precancers, pigmentary disorders, genetic skin disorders, inflammatory skin disorders, infectious skin diseases, wound healing disorders, and scarring disorders.

3. The method of claim 1, wherein the oligonucleotide modulates gene expression by mRNA degradation, translation inhibition, splice modulation, RNA editing, gene activation via RNA activation (RNAa), or any combination thereof.

4. The method of claim 1, wherein the oligonucleotide comprises a sequence complementary to a region of the one or more transcripts associated with the skin-related condition.

5. The method of claim 4, wherein the one or more transcripts comprise a human transcript selected from: interleukin-17A (IL-17A); interleukin-17A receptor (IL-17RA); interleukin-23 (IL-23); interleukin-17F (IL-17F); interleukin-17C receptor (IL-17RC); tumor necrosis factor-alpha (TNF-α); interferon-gamma (IFN-γ); Janus kinase 1 (JAK1); Janus kinase 3 (JAK3); signal transducer and activator of transcription 3 (STAT3); tyrosinase (TYR); microphthalmia-associated transcription factor (MITF); collagen type I alpha 1 (COL1A1); matrix metalloproteinase 9 (MMP9); cyclin dependent kinase inhibitor 2A (CDKN2A); B-Raf proto-oncogene, serine/threonine kinase (BRAF); or any combination thereof.

6. The method of claim 5, wherein the sequence complementary to the region of the disease-associated transcript comprises any one of SEQ ID NOs: 1-10.

7. The method of claim 1, wherein the oligonucleotide comprises a chemically modified nucleic acid.

8. The method of claim 7, wherein the chemically modified nucleic acid comprises one or more locked nucleic acid (LNA) modified bases, one or more phosphorothioate internucleotide linkages, one or more RNA bases with 2′ modifications, or any combination thereof.

9. The method of claim 1, wherein the polymer backbone comprises two or more monomers selected from:
a synthetic monomer selected from a serinol, a norbornene, an acrylate, and an acrylamide;
a natural monomer selected from an amino acid and a sugar;
a modified form of a natural molecule selected from a morpholino phosphorodiamidate, a modified amino acid, a modified spermine, a modified lipid, and a modified cholesterol;
or any combination thereof.

10. The method of claim 1, wherein:
the bottlebrush polymer-oligonucleotide conjugate comprises about 30 PEG polymer arms;
each of the PEG polymer arms is about 10 kDa; or
both of the foregoing.

11. The method of claim 1, wherein the composition is administered to the subject as a systemic injection.

12. The method of claim 11, wherein the composition is administered to the subject as a dose of about 0.5 mg oligonucleotide per kg bodyweight (0.5 mg/kg) to about 5 mg oligonucleotide per kg bodyweight (5 mg/kg).

13. The method of claim 1, wherein the composition is administered to the subject topically, intradermally, or transdermally.

14. The method of claim 1, wherein the bottlebrush polymer-oligonucleotide conjugate further comprises a targeting ligand.

15. A bottlebrush polymer-oligonucleotide conjugate, comprising:
a polymer backbone;
polyethylene glycol (PEG) polymer arms covalently linked to the polymer backbone; and
an oligonucleotide covalently linked to the backbone, wherein the oligonucleotide modulates expression of one or more transcripts associated with the skin-related condition.

16. A composition comprising the bottlebrush polymer-oligonucleotide conjugate of claim 15 and one or more pharmaceutically acceptable excipients, diluents, or carriers suitable for topical, transdermal, intradermal, or systemic administration.

17. A method for diagnosing a skin-related condition in a subject in need thereof, comprising:
(1) scoring or measuring a parameter related to the skin-related condition in the subject, thereby obtaining a pre-treatment level of the parameter;
(2) administering to the subject a therapeutically effective amount of a composition comprising a bottlebrush polymer-oligonucleotide conjugate, wherein the conjugate comprises:
a polymer backbone,
polyethylene glycol (PEG) polymer arms covalently linked to the polymer backbone, and
an oligonucleotide covalently linked to the backbone, wherein the oligonucleotide modulates expression of one or more transcripts associated with the skin-related condition;
(3) scoring or measuring the parameter related to the skin-related condition in the subject, thereby obtaining a post-treatment level of the parameter; and
(4) comparing the post-treatment level to the pre-treatment level, wherein a difference between the post-treatment level and the pre-treatment level indicates presence of the skin-related condition in the subject.

18. A method for improving an aspect of skin of a subject, comprising administering to the subject a composition comprising a bottlebrush polymer-oligonucleotide conjugate, wherein the conjugate comprises:
a polymer backbone;
polyethylene glycol (PEG) polymer arms covalently linked to the polymer backbone; and
an oligonucleotide covalently linked to the backbone, wherein the oligonucleotide modulates expression of one or more transcripts associated with the skin-related condition.

19. The method of claim 18, wherein the aspect comprises one or more of pigmentation irregularities, uneven skin tone, rough or dry skin texture, fine lines, wrinkles, and scars.
